(19) United States
(12) Patent Application Publication
(10) Pub. No.: US 2008/0312326 A1
(43) Pub. Date: Dec. 18, 2008

(54) Method for the Prevention and/or Treatment of Neurodegenerative Diseases Characterized by Administering and Ep1 Receptor Antagonist

(75) Inventor: Sylvain Dore, Baltimore, MD (US)

Correspondence Address: BANNER & WITCOFF, LTD., 1100 13th STREET, N.W., SUITE 1200, WASHINGTON, DC, 20005-4051, US

(73) Assignee: THE JOHN HOPKINS UNIVERSITY, BALTIMORE, MD (US)

(21) Appl. No.: 11/917,058

(22) PCT Filed: May 25, 2006
(86) PCT No.: PCT/US2006/020144

§ 371 Date: Jul. 15, 2008

Related U.S. Application Data
(60) Provisional application No. 60/689,059, filed on Jun. 10, 2005.

(51) Int. Cl. A61K 31/195; A61P 25/00

(52) U.S. Cl. 514/562

(57) ABSTRACT

The present invention relates to the use of EP₁ receptor antagonists for the treatment of neurodegenerative diseases, for example, Alzheimer's disease, Parkinson's disease, Parkinson syndrome, dementia, amyotrophic lateral sclerosis, progressive supranuclear palsy, Huntington's disease, spinocerebellar ataxia, etc.

### FIELD OF THE INVENTION

The present invention relates to a method for the (i) prevention, (ii) treatment and/or (iii) inhibition of progress of neurodegenerative diseases. More particularly, it relates to a method for the (i) prevention, (ii) treatment and/or (iii) inhibition of progress of neurodegenerative diseases in a patient comprising administering a therapeutically effective amount of a compound having an antagonism to an EP₁ receptor.

### BACKGROUND OF THE INVENTION

Prostaglandin E₂ (abbreviated as PGE₂ hereinafter) has been known as a metabolite in an arachidonic acid cascade and has been known to have cyto-protective activity, uterine contractile activity, a pain-inducing effect, a promoting effect on digestive peristalsis, an awakening effect, a suppressive effect on gastric acid secretion, hypotensive activity and diuretic activity, etc.

During the studies in recent years, it has been clarified that, in PGE₂ receptor, there are subtypes having each different role. When roughly classified, there are four subtypes which have been known up to now and each of them is called EP₁, EP₂, EP₃ and EP₄ (J. Lipid Mediators Cell Signaling, 12, 373-391 (1995)).

Since PGE₂ has so many physiologic activities, it has a disadvantage that an effect other than the desired effect turns into a side effect and studies have been continued for overcoming the disadvantage by investigating the role of each subtype so as to prepare a compound which is useful only for the subtype.

Among those subtypes, an EP₁ receptor has been known to be related to a pain-inducing effect, a fever-inducing effect and diuresis (q.v. Br. J. Pharmacol., 1994, 112, 735-740; Eur. J. Pharmacol., 152 (1988) 273-9; and Gen Pharmacol., September 1992, 23 (5), 805-9). However, it is still unclear whether EP₁ receptor is concerned with neurodegenerative diseases.

Cyclooxygenases (COX-1, 2), the rate-limiting enzymes involved in the biosynthesis of prostaglandins, are present constitutively in nearly all cells. Also, COX-2 is an inducible isoform that is strongly induced as a result of pathological events associated with the excessive activation of N-methyl-D-aspartate (NMDA) receptors, such as cerebral ischemia (see J Pharmacol Exp Ther. 2000, 293, 417-425, Proc. Natl. Acad. Sci. USA. 2001, 98, 1294-1299), seizures (J Neurochem. 1996, 66, 6-13), excitotoxic brain injury (J Neurosci Res. 2002, 68, 745-754) besides other neurological disorders. A role for COX-2 in excitotoxicity is validated by the findings that COX-2-deficiency decreases the susceptibility to both ischemic brain injury and NMDA-mediated neurotoxicity in vivo (Proc. Natl. Acad. Sci. USA. 2001, 98, 1294-1299); however, neuronal overexpression of COX-2 in mice shows increased infarction volume (Ann. Neurol. 2003, 54, 155-162). Moreover, treatment of cultured mouse cortical neurons by NMDA induces COX-2 expression, whereas the inhibition of this enzyme was protective against NMDA-induced neurotoxicity (J Pharmacol Exp Ther. 2000, 293, 417-425, J. Neurosci. Res. 2003, 71, 79-88).

The recent medical and media attention drawn by reports of increased cardiovascular malfunction due to use of COX-2 inhibitors led to proscription of these drugs, called into question their safety, and shifted the focus from COX to prostaglandins and their G-protein-coupled receptors. Prostaglandins are known to affect the central nervous system and can modulate synaptic transmission and neurotransmitter release, the sleep/wake cycle, fever, pain and immune system, as well as influence the neuroglial interactions (Physiol Rev. 1999, 79, 1193-1226, Life Sci. 2003, 74, 391-395, Prostaglandins. 1997, 54, 601-624). PGE₂ is one of the important prostaglandins involved in the normal regulation of brain activities, with conflicting data reported regarding its action in brain. These contentious effects of PGE₂ might be the result of its prostanoid receptors: EP₁ to EP₄. EP₂ and EP₄ receptors are involved in intracellular increase in the cAMP levels; while the EP₃ receptors would decrease the cAMP levels and the EP₁ receptors are linked with Ca²⁺ and inositol 1,4,5-triphosphate (IP₃) regulation (Physiol Rev. 1999, 79, 1193-1226). We postulate that in the CNS, EP₁/EP₃ receptors would be responsible for some of the devastating effects of PGE₂.

Neurodegenerative diseases are symptoms of systematic degeneration and drop out of neurons and a lot of severe diseases are known, e.g. Alzheimer's disease, Parkinson's disease, Parkinson syndrome, dementia, amyotrophic lateral sclerosis, progressive supranuclear palsy, Huntington's disease, spinocerebellar ataxia, etc.

A lot of molecules are involved with the mechanism of neurodegenerative death and it is assumed that they are overexpressed or functional disorder occurs in them. However, almost nothing is known on the molecular pathology and an effective method for the treatment is not established yet.

Neurodegenerative diseases symptoms include tremor, rigidity, akinesia, bradykinesia, slow movement, postural reflex disorder, pulsion, gait disorder, depression, dysmnesia, amyotrophy, muscle weakness, dysfunctions of upper extremities, dysarthria, dysphagia, respiratory obstruction, palsy, paralysis, etc.

On the other hand, one subtype of PGE₂ receptor, i.e. EP₂ is known to be concerned with neuroprotection (see Neurobiology of Disease, 24, 1, 257-268, 2004). However, another subtype of PGE₂ receptor EP₁ is not known to be concerned with neuroprotection.

### BRIEF EXPLANATION OF THE FIGURES

FIG. 1 shows the effect of Middle Cerebral Artery Occlusion (MCAO) on Cerebral Blood Flow (CBF) as measured by laser-Doppler flowmetry (LDF).

FIG. 2 shows the effect of MCAO on body temperature.

FIG. 3 shows the Mean Arterial Blood Pressure (MABP) after induction of ischemia.

FIG. 4 shows the infarction volume in a mouse model of MCAO in two groups (wildtype and EP₁^{-/-}).

FIG. 5 shows the lesion volume to NMDA-induced toxicity as compared to wildtype mice and EP₁^{-/-} knockout mice with and without ICV treatment of compound B. The ICV treatment of EP₁^{-/-} mice with compound B produced no significant difference as compared to the EP₁^{-/-} group.

FIG. 6 shows the lesion volume to NMDA-induced toxicity as compared to EP₁^{-/-} mice with WT mice and with and without ICV treatment of compound A (agonist).

FIG. 7 shows the lesion volume to NMDA-induced toxicity as compared to pretreatment with compound B (antagonist).

### DISCLOSURE OF THE INVENTION

The inventors have carried out intensive investigations for finding compounds which are effective for the treatment and/or the prevention of neurodegenerative diseases. Compounds having an antagonism to an EP₁ receptor when applied to neurodegenerative diseases-inducing model of animals suppress the neurodegenerative diseases. Previously, no relation was shown between subtypes of PGE₂ receptors and the specific diseases and no relation between EP₁ receptor antagonism and neurodegenerative diseases was suggested.

Thus, the present invention provides a method for the protection of neurons. More particularly, the present invention provides a method for the protection of neurons, characterized by administering to a mammal an effective amount of EP₁ receptor antagonist, and a pharmaceutical composition comprising an EP₁ receptor antagonist.

The present invention relates to

[1] a method for the treatment and/or prevention of neurodegenerative diseases in a patient comprising administering a therapeutically effective amount of a compound having an antagonism to an EP₁ receptor.
[2] the method according to above [1], wherein the compound having an antagonism to an EP₁ receptor is selected from

### (1) A Compound of Formula (A)

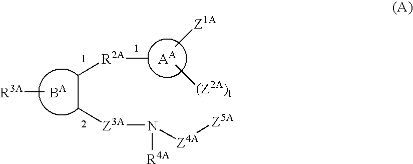

(wherein

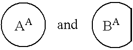

each independently is a C₅₋₁₅ carbon ring or a five- to seven-membered hereto ring having 1 or 2 oxygen, sulfur or nitrogen atom(s),
Z^{1A} is —COR^{1A}, —C₁₋₄ alkylene-COR^{1A}, —CH═CH—COR^{1A}, —C≡C—COR^{1A}, —O—C₁₋₃ alkylene-COR^{1A} (in each formula R^{1A} is hydroxyl group, C₁₋₄ alkoxy or a group represented by a formula NR^{6A}R^{7A} (in the formula R^{6A} and R^{7A} each independently is a hydrogen atom or C₁₋₄ alkyl) or —C₁₋₅ alkylene-OH,

Z^{2A} is a hydrogen, C₁₋₄ alkyl, C₁₋₄ alkoxy, nitro, halogen, trifluoromethyl, trifluoromethoxy, a hydroxyl group or a group represented by the formula COR^{1A} (in the formula R^{1A} has the same meaning as hereinbefore defined),

Z^{3A} is a single bond or C₁₋₄ alkylene,

### Z^{4A} is SO₂ or CO,

### Z^{5A} is

(1) C₁₋₈ alkyl, C₂₋₈ alkenyl, C₂₋₈ alkynyl,
(2) phenyl, C₃₋₇ cycloalkyl, a five- to seven-membered hetero ring having one or two oxygen, sulfur or nitrogen atom(s),
(3) phenyl or C₃₋₇ cycloalkyl-substituted C₁₋₄ alkyl, C₂₋₄ alkenyl or C₂₋₄ alkynyl,
(in the above (2) and (3), phenyl, C₃₋₇ cycloalkyl and a five- to seven-membered hetero ring having one or two oxygen, sulfur or nitrogen atom(s) may be substituted with one to five R^{5A} group(s) (each of plural R^{5A} independently is a hydrogen atom, C₁₋₆ alkyl, C₁₋₆ alkoxy, C₁₋₆ alkylthio, nitro, halogen, trifluoromethyl, trifluoromethoxy or a hydroxyl group)),
R^{2A} is CONR^{8A}, NR^{8A}CO, CONR^{8A}—C₁₋₄ alkylene, C₁₋₄ alkylene-CONR^{8A}, NR^{8A}CO—C₁₋₄ alkylene, C₁₋₄ alkylene-NR^{8A}CO, C₁₋₃ alkylene-CONR^{8A}—C₁₋₃ alkylene, C₁₋₃ alkylene-NR^{8A}CO—C₁₋₃ alkylene (in each formula, R^{8A} is a hydrogen atom or C₁₋₄ alkyl), O, S, NZ^{6A} (in each formula, Z^{6A} is a hydrogen atom or C₁₋₄ alkyl), Z^{7A}-C₁₋₄ alkylene, C₁₋₄ alkylene-Z^{7A}, C₁₋₃ alkylene-Z^{7A}-C₁₋₃ alkylene (in the formulae, Z^{7A} is O, S or a group represented by the formula NZ^{6A} (in the formula, Z^{6A} has the same meaning as defined above)), CO, CO—C₁₋₄ alkylene, C₁₋₄ alkylene-CO, C₁₋₃ alkylene-CO—C₁₋₃ alkylene, C₂₋₄ alkylene, C₂₋₄ alkenylene or C₂₋₄ alkynylene,
R^{3A} is a hydrogen atom, C₁₋₆ alkyl, C₁₋₆ alkoxy, C₁₋₆ alkylthio, nitro, halogen, trifluoromethyl, trifluoromethoxy, hydroxy or hydroxymethyl,

### R^{4A} is

(1) a hydrogen atom,
(2) C₁₋₈ alkyl, C₂₋₈ alkenyl, C₂₋₈ alkynyl,
(3) C₁₋₆ alkyl substituted with one or two group(s) selected from the group consisting of COOZ^{8A}, CONZ^{9A}Z^{10A}, OZ^{8A} (in each group, Z^{8A}, Z^{9A} and Z^{10A} each independently is a hydrogen atom or C₁₋₄ alkyl), C₁₋₄ alkoxy-C₁₋₄ alkoxy,
(4) C₃₋₇ cycloalkyl,
(5) phenyl or C₃₋₇ cycloalkyl-substituted C₁₋₄ alkyl, C₂₋₄ alkenyl or C₂₋₄ alkynyl, (phenyl and C₃₋₇ cycloalkyl in the above (4) and (5) may be substituted with one to five R^{5A} group(s) (R^{5A} has the same meaning as defined above))
and n^{A} and t^{A} each independently is an integer of 1 to 4,
wherein
(1) R^{2A} and Z^{3A} each binds to only 1- and 2-position of

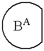

and
(2) when

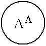

is a benzene ring and (Z^{2A})ₜ^{A} is not COR^{1A}, then Z^{1A} binds to only 3- or 4-position of the benzene ring) or a non-toxic salt thereof,

### (2) A Compound of Formula (B)

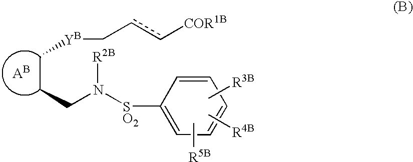

(in the formula,

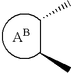

is a group represented by

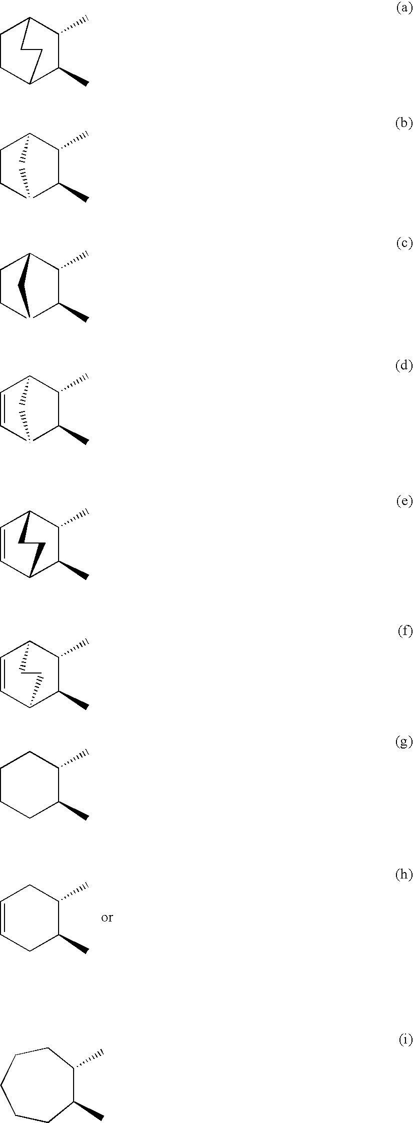

R^{1B} is hydroxy, C₁₋₄ alkoxy or alkoxy or a group represented by formula NR^{6B}R^{7B} (in the formula, R^{6B} and R^{7B} each independently is a hydrogen atom or C₁₋₄ alkyl group),

R^{2B} is a hydrogen atom or C₁₋₄ alkyl group,

R^{3B} and R^{4B} are a C₁₋₄ alkyl group, a halogen atom or trifluoromethyl group,

R^{5B} is a hydrogen atom, a C1-4 alkyl group, a halogen atom or trifluoromethyl group,

Y^{B} is cis-vinylene or trans-vinylene and

a symbol [symbol] is a single bond or a double bond,

wherein when

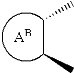

is a formula

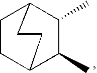

R^{1B} is hydroxy or C₁₋₄ alkoxy group, R^{2B} is a hydrogen atom, Y^{B} is cis-vinylene and the symbol [symbol] is a single bond, then

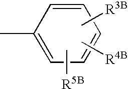

is not

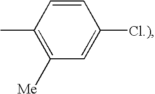

or a non-toxic salt thereof or a cyclodextrin clathrate thereof and

### (3) A Compound of Formula (H)

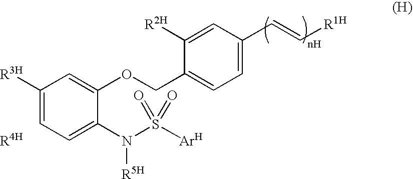

(wherein R^{1H} is COOH, 5-tetrazolyl, 5-oxo-1,2,4-oxadiazolyl, CH₂OH or 5-oxo-1,2,4-thiadiazolyl,
R^{2H} is hydrogen, methyl, methoxy or chloro,
R^{3H} and R^{4H} are the combination of (1) methyl and methyl, (2) methyl and chloro, (3) chloro and methyl, (4) trifluoromethyl and hydrogen, or
R^{3H} and R^{4H} are taken together to form cyclopentene, (6) cyclohexene or (7) benzene,
Ar^{H} is thiazolyl optionally substituted with methyl, pyridyl or 5-methyl-2-furyl,
nH is 0 or 1, but when R^{1H} is 5-tetrazolyl, 5-oxo-1,2,4-oxazolyl or 5-oxo-1,2,4-thiazolyl, nH is 0), its alkyl ester or its non-toxic salt,
[3] a pharmaceutical composition comprising a compound having an antagonism to an EP₁ receptor for the treatment, prevention and/or inhibition of progress of neurodegenerative diseases,
[4] the pharmaceutical composition according to above [3], wherein the neurodegenerative disease is selected from Parkinson's disease, Parkinson syndrome, Alzheimer's disease, dementia, amyotrophic lateral sclerosis and cerebral stroke,
[5] the pharmaceutical composition according to above [3], characterized by ameliorating the symptoms of one neurodegenerative disease or more selected from tremor, rigidity, akinesia, bradykinesia, slow movement, postural reflex disorder, pulsion, gait disorder, depression, dysmnesia, amyotrophy, muscle weakness, dysfunctions of upper extremities, dysarthria, dysphagia, respiratory obstruction, palsy, paralysis, etc. which are symptoms of neurodegenerative diseases,
[6] a method for protecting neurons induced by an excitory amino acid comprising administering an EP₁ receptor antagonist,
[7] the pharmaceutical composition according to above [3], wherein the EP₁ receptor antagonist is administered together with a dopamine receptor agonist, monoamine oxidase inhibitor, COMT inhibitor, acetylcholine esterase inhibitor, β-amyloid protein aggregation inhibitor, β-secretase inhibitor, brain function activator, antioxidant, antithrombotic and astrocyte ameliorator and NMDA receptor antagonist.
[8] the pharmaceutical composition wherein the EP₁ receptor antagonist is selected from
(1) (5Z)-6-[(2R,3S)-3-({[(4-chloro-2-methylphenyl)sulfonyl]amino}methyl)bicycle [2.2.2]oct-2-yl]hex-5-enoic acid,
(2) (2E)-3-(4-{[2-[(2-furylsulfonyl)(isobutyl)amino]-5-(trifluoromethyl)phenoxy]methyl}phenyl)acrylic acid,
(3) 4-{[2-{isopropyl[(5-methyl-2-furyl)sulfonyl]amino}-5-(trifluoromethyl)phenoxy]methyl}benzoic acid
(4) 4-{[(6-{isobutyl[(4-methyl-1,3-thiazol-2-yl)sulfonyl]amino}-2,3-dihydro-1H-inden-5-yl)oxy]methyl}benzoic acid, a salt thereof, an N-oxide or solvate thereof, a prodrug thereof, or a cyclodextrin clathrate thereof,
   [9] use of an EP₁ receptor antagonist for the manufacture of a medicament for the prevention, treatment and/or inhibition of progress of neurodegenerative disease and
   [10] a method for the inhibition of progress of neurodegenerative diseases in a patient comprising administering a therapeutically effective amount of a compound having an antagonism to an EP₁ receptor.

Compounds having an antagonism to an EP₁ receptor used in the present invention include not only EP₁ receptor antagonists which have been known but also any EP₁ receptor antagonists which will be found in the future. The following compounds are preferably used (Details of the definitions of symbols in the formulae shown hereinafter are the same as those mentioned in each of the specifications, the disclosures of which are expressly incorporated herein).

### (1) A Compound of Formula (A) Mentioned in WO 98/27053

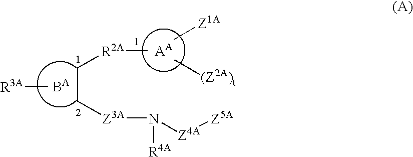

(wherein

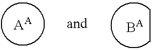

each independently is a C₅₋₁₅ carbon ring or a five- to seven-membered hereto ring having 1 or 2 oxygen, sulfur or nitrogen atom(s),
Z^{1A} is —COR^{1A}, —C₁₋₄ alkylene-COR^{1A}, —CH═CH—COR^{1A}, —C≡C—COR^{1A}, —O—C₁₋₃ alkylene-COR^{1A} (in each formula R^{1A} is hydroxyl group, C₁₋₄ alkoxy or a group represented by a formula NR^{6A}R^{7A} (in the formula R^{6A} and R^{7A} each independently is a hydrogen atom or C₁₋₄ alkyl) or —C₁₋₅ alkylene-OH,

Z^{2A} is a hydrogen, C₁₋₄ alkyl, C₁₋₄ alkoxy, nitro, halogen, trifluoromethyl, trifluoromethoxy, a hydroxyl group or a group represented by the formula COR^{1A} (in the formula R^{1A} has the same meaning as hereinbefore defined),

Z^{3A} is a single bond or C₁₋₄ alkylene,

### Z^{4A} is SO₂ or CO,

### Z^{5A} is

(1) C₁₋₈ alkyl, C₂₋₈ alkenyl, C₂₋₈ alkynyl,
(2) phenyl, C₃₋₇ cycloalkyl, a five- to seven-membered hetero ring having one or two oxygen, sulfur or nitrogen atom(s),
(3) phenyl or C₃₋₇ cycloalkyl-substituted C₁₋₄ alkyl, C₂₋₄ alkenyl or C₂₋₄ alkynyl, (in the above (2) and (3), phenyl, C₃₋₇ cycloalkyl and a five- to seven-membered hetero ring having one or two oxygen, sulfur or nitrogen atom(s) may be substituted with one to five R^{5A} group(s) (each of plural R^{5A} independently is a hydrogen atom, C₁₋₆ alkyl, C₁₋₆ alkoxy, C₁₋₆ alkylthio, nitro, halogen, trifluoromethyl, trifluoromethoxy or a hydroxyl group)),
R^{2A} is CONR^{8A}, NR^{8A}CO, CONR^{8A}—C₁₋₄ alkylene, C₁₋₄ alkylene-CONR^{8A}, NR^{8A}CO—C₁₋₄ alkylene, C₁₋₄ alkylene-NR^{8A}CO, C₁₋₃ alkylene-CONR^{8A}—C₁₋₃ alkylene, C₁₋₃ alkylene-NR^{8A}CO—C₁₋₃ alkylene (in each formula, R^{8A} is a hydrogen atom or C₁₋₄ alkyl), O, S, NZ^{6A} (in each formula, Z^{6A} is a hydrogen atom or C₁₋₄ alkyl), Z^{7A}-C₁₋₄ alkylene, C₁₋₄ alkylene-Z^{7A}, C₁₋₃ alkylene-Z^{7A}-C₁₋₃ alkylene (in the formulae, Z^{7A} is O, S or a group represented by the formula NZ^{6A} (in the formula, Z^{6A} has the same meaning as defined above)), CO, CO—C₁₋₄ alkylene, C₁₋₄ alkylene-CO, C₁₋₃ alkylene-CO—C₁₋₃ alkylene, C₂₋₄ alkylene, C₂₋₄ alkenylene or C₂₋₄ alkynylene,
R^{3A} is a hydrogen atom, C₁₋₆ alkyl, C₁₋₆ alkoxy, C₁₋₆ alkylthio, nitro, halogen, trifluoromethyl, trifluoromethoxy, hydroxy or hydroxymethyl,

### R^{4A} is

(1) a hydrogen atom,
(2) C₁₋₈ alkyl, C₂₋₈ alkenyl, C₂₋₈ alkynyl,
(3) C₁₋₆ alkyl substituted with one or two group(s) selected from the group consisting of COOZ^{8A}, CONZ^{9A}Z^{10A}, OZ^{8A} (in each group, Z^{8A}, Z^{9A} and Z^{10A} each independently is a hydrogen atom or C₁₋₄ alkyl), C₁₋₄ alkoxy-C₁₋₄ alkoxy,
(4) C₃₋₇ cycloalkyl,
(5) phenyl or C₃₋₇ cycloalkyl-substituted C₁₋₄ alkyl, C₂₋₄ alkenyl or C₂₋₄ alkynyl, (phenyl and C₃₋₇ cycloalkyl in the above (4) and (5) may be substituted with one to five R^{5A} group(s) (R^{5A} has the same meaning as defined above))
and n^{A} and t^{A} each independently is an integer of 1 to 4,
wherein
(1) R^{2A} and Z^{3A} each binds to only 1- and 2-position of

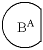

and
(2) when

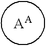

is a benzene ring and (Z^{2A})ₜ^{A} is not COR^{1A}, then Z^{1A} binds to only 3- or 4-position of the benzene ring) or a non-toxic salt thereof.

### (2) A Compound of Formula (B) Mentioned in EP 878465

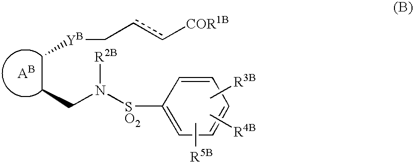

(in the formula,

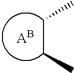

is a group represented by

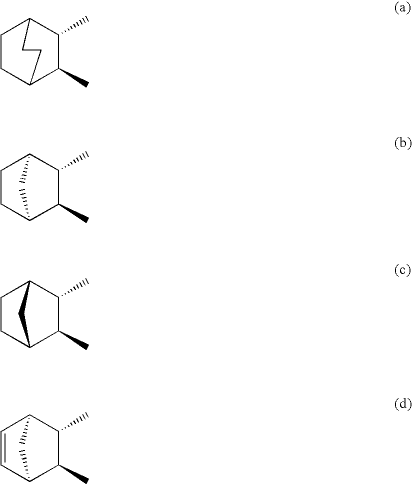

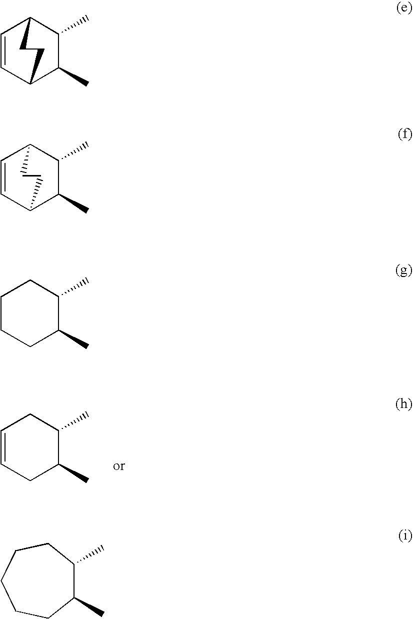

R^{1B} is hydroxy, C₁₋₄ alkoxy or alkoxy or a group represented by formula NR^{6B}R^{7B} (in the formula, R^{6B} and R^{7B} each independently is a hydrogen atom or C₁₋₄ alkyl group),

R^{2B} is a hydrogen atom or C₁₋₄ alkyl group,

R^{3B} and R^{4B} are a C₁₋₄ alkyl group, a halogen atom or trifluoromethyl group,

R^{5B} is a hydrogen atom, a C₁₋₄ alkyl group, a halogen atom or trifluoromethyl group,

Y^{B} is cis-vinylene or trans-vinylene and

a symbol [symbol] is a single bond or a double bond,

wherein when

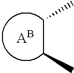

is a formula

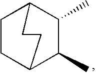

R^{1B} is hydroxy or C₁₋₄ alkoxy group, R^{2B} is a hydrogen atom, Y^{B} is cis-vinylene and the symbol [symbol] is a single bond, then

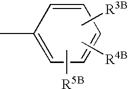

is not

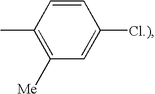

or a non-toxic salt thereof or a cyclodextrin clathrate thereof.

### (3) A Compound of Formula (C) Mentioned in the Specification of WO 92/19617

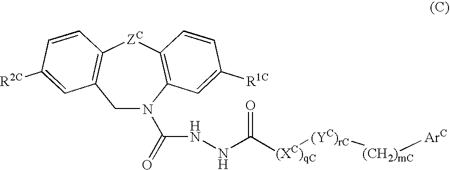

(in the formula, R^{1C} is a hydrogen atom, halogen or —CF₃;
R^{2C} is a hydrogen atom, halogen, —OH or —OCH₃;

### Z^{C} is —O—, —S—, —S(O)— or —S(O)₂—;

X^{C} is —CH═CH—, —CF₂—, —CHF—, —(CH₂)_{nC}— or —(CH₂)_{pC}—CH═CH—;

### Y^{C} is, —CH(OH)—, —NR^{3C}—, —S—, —S(O)—, —S(O)₂— or —O—;

q^{C} is 0 or 1;
r^{C} is 0 or 1 (wherein when
(1) X^{C} is —CH═CH—, —(CH₂)_{nC}— or —(CH₂)_{pC}—CH═CH—, q^{C} is 1, and Ar^{C} is imidazole or phenyl,
(2) X^{C} is —(CH₂)_{nC}—, q^{C} is 1, n^{C} is 1, and Ar^{C} is halogen, methyl or alkoxy-substituted ethylphenyl, or
(3) q^{C} is 1, m^{C} is 1, 2, 3, 4, 5 or 6, and Ar^{C} is imidazolyl or phenyl, then r^{C} is not 0);
m^{C} is 0 to 6 (wherein when X^{C} is —(CH₂)_{nC}—, q^{C} is 1, Y^{C} is —O—, —S—, —S(O)— or —S(O)₂—, and
Ar^{C} is phenyl, m^{C} is not 0);
n^{C} is an integer of 1 to 6;
p^{C} is an integer of 1 to 6;
R^{3C} is a hydrogen atom or t-butyloxycarbonyl, and
Ar^{C} is aryl, alkyl-substituted aryl or aryl-substituted aryl).

### (4) A Compound of Formula (D) Mentioned in the Specification of WO 96/06822

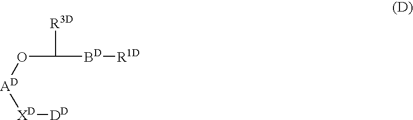

(in the formula, A^{D} is optionally substituted eight-to ten membered bicyclic heteroaryl, five- or six-membered heteroaryl, naphthyl, or phenyl where the binding groups —OCH(R^{3D})— and —X^{D}— are positioned at 1- and 2-positions each other on a cyclic carbon atom,
B^{D} is optionally substituted five- or six-membered heteroaryl ring or optionally-substituted phenyl,
D^{D} is optionally substituted pyridyl, pyrazinyl, pyrimidinyl, pyridazinyl, pyrrolyl, thienyl, furyl, pyrazolyl, thiazolyl, isothiazolyl, oxazolyl, isoxazolyl or phenyl,
X^{D} is, a formula represented by —(CHR^{4D})_{nD}— or —(CHR^{4D})_{pD}CR^{4D}═CR^{4D}(CHR^{4D})_{qD}— in which n^{D} is 1 to 3 and both p^{D} and q^{D} are 0 or one of p^{D} and q^{D} is 1 while the other is 0,
R^{1D} is positioned on the ring BD in a relation of 1, 3 or 1,4 with a binding group —OCH(R^{3D})— on a six-membered ring or in a relation of 1,3- with a binding group —OCH(R^{3D})— on a five-membered ring and is carboxy, carboxy-C₁₋₃-alkyl, tetrazolyl, tetrazolyl-C1-C3-alkyl, tetronic acid, hydroxamic acid, sulfonic acid, or R^{1D} is a formula —CONR^{aD}R^{a1D} in which R^{aD} is a hydrogen atom or C₁₋₆ alkyl, R^{a1} is a hydrogen atom or optionally substituted C₁₋₆ alkyl, C₂₋₆ alkenyl, C₂₋₆ alkynyl, C₃₋₇ cycloalkyl, C₃₋₇ cycloalkyl-C₁₋₆ alkyl, C₃₋₇ cycloalkyl-C₂₋₆ alkenyl, C₃₋₇ cycloalkyl-C₂₋₆ alkynyl, C₅₋₇ cycloalkenyl, C₃₋₇ cycloalkenyl-C₁₋₆alkyl, C₅₋₇ cycloalkenyl-C₂₋₆ alkenyl, C₅₋₇ cycloalkenyl-C₂₋₆ alkynyl, C₁₋₃ alkyl which is substituted with a five- or six-membered saturated or partially saturated heteroring, five- or six-membered saturated or partially saturated heteroring, five- or six-membered heteroaryl or, in the formula, R^{aD} and R^{a1D} form an amino acid residue or ester thereof together with an amide nitrogen (NR^{aD}R^{a1D}) to which they bind, or R^{1D} is a formula —CONHSO₂R^{bD} in which R^{bD} is optionally substituted C₁₋₆ alkyl, C₂₋₆ alkenyl, C₂₋₆ alkynyl, C₃₋₇ cycloalkyl-C₁₋₆ alkyl, C₃₋₇ cycloalkyl-C₂₋₆ alkenyl, C₃₋₇ cycloalkyl-C₂₋₆ alkynyl, C₃₋₇ cycloalkenyl-C₁₋₆ alkyl, C₃₋₇ cycloalkenyl-C₂₋₆ alkenyl, C₃₋₇ cycloalkenyl-C₂₋₆ alkynyl, five- or six-membered heteroaryl, five- or six-membered heteroaryl-C₁₋₆ alkyl, phenyl, phenyl-C₁₋₆ alkyl, five- or six-membered saturated or partially saturated heteroring or five- or six-membered saturated or partially saturated heteroring-C₁₋₆ alkyl, R^{3D} is a hydrogen atom or C₁₋₄ alkyl,
R^{4D} is a hydrogen atom or C₁₋₄ alkyl), or an N-oxide thereof in case chemically possible, a sulfur oxide having ring in cade chemically possible, a pharmaceutically acceptable salt thereof, or an ester or amide hydrolysable in the living body.

### (5) A Compound of Formula (E) Mentioned in WO 97/00863

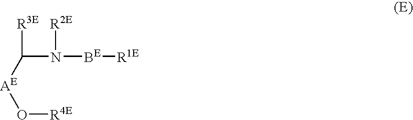

(in the formula, A^{E} is the following which is optionally substituted: phenyl, naphthyl, pyridyl, pyrazinyl, pyridazinyl, pyrimidinyl, thienyl, thiazolyl, oxazolyl or thiadiazolyl having at least two adjacent ring carbon atoms;
in that case, —CH(R^{3E})N(R^{2E})B^{E}—R^{1E} and OR^{4E} are positioned at 1 and 2 each other on the ring carbon atoms and the ring atom positioned at ortho to an OR^{4E} binding group (and, therefore, at 3-position on the basis of a —CHR^{3E}NR^{2E}— binding group) is not substituted;
B^{E} is the following which is optionally substituted:
phenyl, pyridyl, thiazolyl, oxazolyl, thienyl, thiadiazolyl, imidazolyl, pyrazinyl, pyridazinyl or pyrimidyl;
R^{1E} is positioned 1, 3 or 1,4 to a —CH(R^{3E})N(R^{2E})— binding group on a ring B^{E} and R^{1E} is carboxy, carboxy-C₁₋₃ alkyl, tetrazolyl, tetrazolyl C₁₋₃ alkyl, tetronic acid, hydroxamic acid, sulfonic acid or R^{1E} is —CONR^{aE}R^{a1E} [in that case, R^{aE} is a hydrogen atom or C₁₋₆ alkyl, and R^{a1E} is a hydrogen atom, C₁₋₆ alkyl (in some cases, it is substituted with halogen, amino, C₁₋₄ alkylamino, di-C₁₋₄ alkylamino, hydroxy, nitro, cyano, trifluoromethyl, C₁₋₄ alkoxy or C₁₋₄ alkoxycarbonyl), C₂₋₆ alkenyl (in that case, a double bond is not at 1-position), C₂₋₆ alkynyl (in that case, a triple bond is not at 1-position), carboxyphenyl, five- or six-membered heterocyclyl-C₁₋₃ alkyl, five- or six-membered heteroaryl-C₁₋₃ alkyl, five- or six-membered heterocyclyl or five- or six-membered heteroaryl, or R^{aE} and R^{a1E} form an amino acid residue or ester thereof together with an amide nitrogen (NR^{aE}R^{a1E}) to which they bind] or R¹ is a group of formula —CONHSO₂R^{b} [in that case R^{bE} is C₁₋₆ alkyl (in some cases, it may be substituted with halogen, hydroxy, nitro, cyano, trifluoromethyl, C₁₋₄ alkoxy, amino, C₁₋₄ alkylamino, di-C₁₋₄ alkylamino or C₁₋₄ alkoxycarbonyl), C₂₋₆ alkenyl (in that case, a double bond is not at 1-position), C₂₋₆ alkynyl (in that case, a triple bond is not at 1-position), five- or six-membered heterocyclyl-C₁₋₃ alkyl, five- or six-membered heteroaryl-C₁₋₃ alkyl, five- or six-membered heterocyclyl, five- or six-membered heteroaryl or phenyl];
in that case, any heterocyclyl or heteroaryl group in R^{a1E} is optionally substituted with halogen, hydroxy, nitro, hydroxy, amino, cyano, C₁₋₆ alkoxy, C₁₋₆ alkylS(O)ₚ^{E}— (p^{E} is 0, 1 or 2), C₁₋₆ alkylcarbamoyl, C₁₋₄ alkylcarbamoyl, di(C₁₋₄ alkyl)carbamoyl, C₂₋₆ alkenyl, C₂₋₆ alkynyl, C₁₋₄ alkoxycarbonylamino, C₁₋₄ alkanoylamino, C₁₋₄ alkanoyl(N—C₁₋₄ alkyl)amino, C₁₋₄ alkanesulfonamide, benzenesulfonamide, aminosulfonyl, C₁₋₄ alkylaminosulfonyl, di(C₁₋₄ alkyl)aminosulfonyl, C₁₋₄ alkoxycarbonyl, C₁₋₄ alkanoyloxy, C₁₋₆ alkanoyl, formyl-C₁₋₄ alkyl, hydroxyimino-C₁₋₆ alkyl, C₁₋₄ alkoxyimino-C₁₋₆alkyl or C₁₋₆ alkylcarbamoylamino; or
R^{1E} is a group of formula —SO₂N(R^{CE})R^{C1E} [in that case, R^{CE} is a hydrogen atom or C₁₋₄ alkyl, and R^{C1E} is a hydrogen atom or C₁₋₄ alkyl]; or r¹ is a group of the following formula (E^{A}), (E^{B}) or (E^{C}):

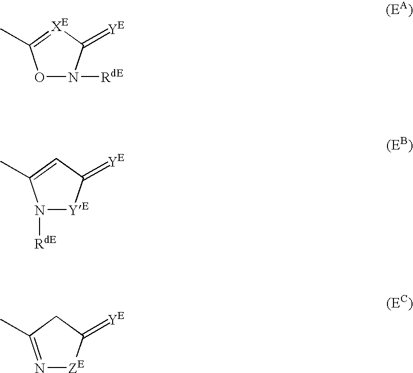

and, in the above formulae, X^{E} is CH or a nitrogen atom, Y^{E} is an oxygen atom or a sulfur atom, Y^{′E} is an oxygen atom or NR^{dE} and Z^{E} is CH₂, NR^{dE} or an oxygen atom and, in that case, there is one or less ring oxygen and there are at least two ring hetero atoms and, in the above formulae, R^{dE} is a hydrogen atom or C₁₋₄ alkyl;
R^{2E} is a hydrogen atom or optionally hydroxy-, cyano- or trifluoromethyl-substituted C₁₋₆ alkyl, C₂₋₆ alkenyl (in that case, a double bond is not at 1-position), C₂₋₆ alkynyl (in that case, a triple bond is not at 1-position), phenyl C₁₋₃ alkyl or pyridyl C₁₋₃ alkyl;
R^{3E} is a hydrogen atom, methyl or ethyl;
R^{4E} is an optionally substituted following:
C₁₋₆ alkyl, C₃₋₇ cycloalkyl C₁₋₃ alkyl or C₃₋₇ cycloalkyl), or an N-oxide of —NR^{2E}— in case chemically possible, or an S-oxide of a sulfur-containing ring in case chemically possible or a pharmaceutically acceptable salt thereof or a hydrolysable ester or amide in the living body.

### (6) A Compound of Formula (F) Mentioned in the Specification of WO 99/47497

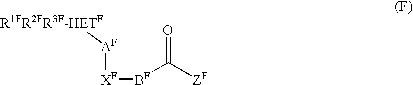

(in the formula, HET^{F} is a five- to twelve-membered monocyclic or a bicyclic aromatic ring containing 0 to 3 hetero atom(s) selected from O, S(O)_{nF} and N(O)_{mF} in which m^{F} is 0 or 1 and n; is 0, 1 or 2,
A^{F} is one or two atomic moiety(ies) and is —W^{F}—, —C(O)—, —C(R^{7F})—W^{F}—, —W^{F}—C(R^{7F})₂—, —CR^{7F}(OR^{20F}), —C(R^{7F})₂—, —C(R^{7F})₂—C(OR^{20F})R^{7F}—, —C(R^{7F})₂—C(R^{7F})₂—, or —CR^{7F}═CR^{7F}—, W^{F} is O, S(O)_{nF} or NR^{17F},
X^{F} is five- to ten-membered monocyclic, bicyclic aryl or five- to ten-membered monocyclic or bicyclic heteroaryl having 1 to 3 hetero atom(s) selected from O, S(O)_{nF} and N(O)_{mF}, which may be substituted with R^{14F} and R^{15F}, where A^{F} and B^{F} bind to ortho position of aryl or heteroaryl,
B^{F} is —(C(R^{18F})₂)_{pF}—Y^{F}—(C(R^{18F})₂)_{qF}—, p^{F} and q^{F} each independently is 0 to 3,
Y^{F} is O, S(O)_{nF}, NR^{17F}, a single bond or —CR^{18F}═CR^{18F}— and, when Y^{F} is O, S(O)_{nF}, NR^{17F} or —CR^{18F}═CR^{18F}—, p^{F}+q^{F} is 0 to 6 while, when Y^{F} is a single bond, p^{F}+q^{F} is 1 to 6,

### Z^{F} is OH or NHSO₂R^{19F},

R^{1F}, R^{2F} and R^{3F} each independently is H, a halogen atom, lower alkyl, lower alkenyl, lower alkynyl, lower alkenyl-HET^{F}(R^{aF})₄₋₉—, —(C(CR^{4F})₂)_{pF})SR^{5F}, —(C(R^{4F})₂)^{pF}OR^{8F}, —(C(R^{4F})₂)_{pF}N(R^{6F})₂, CN, NO₂, —(C(R^{4F})₂)_{pF}C(R^{7F})₃, —COOR^{9F}, —CON(R^{6F})₂ or —(C(R^{4F})₂)_{pF}SS(O)_{nF}R^{10F},
each R^{4F} is H, F, CF₃, lower alkyl, or
two R^{4F} are in conjunction and represent an at most six membered ring which may have one heteroatom selected from O, S(O)_{nF} and N(O)_{mF},
each R^{5F} independently is lower alkyl, lower alkenyl, lower alkynyl, CF₃, lower alkyl-HET^{F}, lower alkenyl-HET^{F}, —(C(R^{18F})₂)_{pF}Ph(R^{11F})₀₋₂,
each R^{6F} independently is H, lower alkyl, lower alkenyl, lower alkynyl, CF₃, phenyl, benzyl or two R^{6F} binding to N are taken in conjunction and represent a at most six membered ring which may have additional heteroatom selected from O, S(O)_{nF} and N(O)_{mF},
each R^{7F} independently is H, F, CF₃, lower alkyl or two R^{7F} are taken in conjunction and represent three- to six-membered aromatic or an aliphatic ring containing 0 to 2 heteroatom(s) selected from O, S(O)_{nF} and N(O)_{mF},
each R^{8F} is H or R^{5F},
each R^{9F} independently is H, lower alkyl, lower alkenyl, lower alkynyl, phenyl or benzyl,
each R^{10F} independently is lower alkyl, lower alkenyl, lower alkynyl, CF₃, Ph(R^{11F})₀₋₃, CH₂Ph(R^{11F})₀₋₃ or N(R^{6F})₂,
each R^{11F} independently is lower alkyl, SR^{20F}, OR^{20F}, N(R^{6F})₂, —COOR^{12F}, —CON(R^{6F})₂, —COR^{12F}, CN, CF₃, NO₂ or a halogen atom,
each R^{12F} independently is H, lower alkyl or benzyl,
each R^{13F} independently is H, a halogen atom, lower alkyl, O-lower alkenyl, S-lower alkyl, N(R^{6F})₂, COOR^{12F}, CN, CF₃ or NO₂,
R^{14F} and R^{15F} independently is lower alkyl, a halogen atom, CF₃, OR^{16F}, S(O)_{nF}R^{16F} or C(R¹⁶)₂OR^{17F},
each R^{16F} independently is H, lower alkyl, lower alkenyl, phenyl, benzyl or CF₃,
each R^{17F} independently is H, lower alkyl or benzyl,
each R^{18F} independently is H, F or lower alkyl, or two R^{18F} are taken in conjunction and represent a three- to six-membered ring which may contain one hetero atom selected from O, S(O)_{nF} and N,
each R^{19F} independently is lower alkyl, lower alkenyl, lower alkynyl, CF₃, HET(R^{aF})₄₋₉, lower alkyl-HET(R^{aF})₄₋₉, lower alkenyl-HET(R^{aF})₄₋₉,
each R^{20F} independently is H, lower alkyl, lower alkenyl, lower alkynyl, CF₃ or Ph(R^{13F})₂,
each R^{aF} independently is a group selected from the followings:
H, OH, a halogen atom, CN, NO₂, amino, C₁₋₆ alkyl, C₂₋₆ alkenyl, C₂₋₆ alkynyl, C₁₋₆ alkoxy, C₂₋₆ alkenyloxy, C₂₋₆ alkynyloxy, C₁₋₆ alkylamino, di(C₁₋₆ alkyl)amino, CF₃, C(O)C₁₋₆ alkyl, C(O)C₂₋₆ alkenyl, C(O)C₂₋₆ alkynyl, COOH, COO—C₁₋₆ alkyl, COO—C₂₋₆ alkenyl and COO—C₂₋₆ alkynyl;
in the group, alkyl, alkenyl, alkynyl, and alkyl in alkylamino or dialkylamino may be substituted with one to three of the following group(s);
OH, a halogen atom, aryl, C₁₋₆ alkoxy, C₂₋₆ alkenyloxy, C₂₋₆ alkynyloxy, CF₃, CO(C₁₋₆)alkyl, CO—C₂₋₆ alkenyl, CO—C₂₋₆ alkynyl, COOH, COO—C₁₋₆ alkyl, COO—C₂₋₆ alkenyl, COO—C₂₋₆ alkynyl, NH₂, NH—C₁₋₆ alkyl and N(C₁₋₆ alkyl)₂), or a non-toxic salt thereof.

### (7) A Compound of Formula (G) Described in WO 00/20371

Ar^{1G}—W^{G}—Ar^{2G}—X^{G}—W^{G} (G)

(in the formula, Ar^{1G} is aryl or heteroaryl, and may be substituted with R^{1G} or R^{3G}, R^{1G} is Y^{G}_{mG}—R^{2G}, Y^{G}_{mG}—Ar^{3G}, a halogen atom, N(R^{5G})₂, CN, NO₂, C(R^{6G})₃, CON(R^{5G})₂, S(O)_{nG}R^{7G}, or OH,
Y^{G} is a connecting chain between R^{2G} or Ar^{3G} and Ar^{1G} and contains 0 to 4 carbon atom(s) and at most one hetero atom selected from O, N and S, and the connecting chain may contain CO, S(O)_{nG}, —C═C— or acetylene or may be further substituted with R^{2G},
m^{G} is 0 or 1, n^{G} is 0, 1 or 2,
R^{2G} is H, F, CHF₂, CF₃, lower alkyl or hydroxy-C₁₋₆ alkyl, or two R^{2G} are taken in combination with an at most six membered ring containing carbon atoms and at most two hetero atom(s) selected from O, N and S,
Ar^{3G} is aryl or heteroaryl which may be substituted with R^{3GG},
R^{3G} is R^{4G}, a halogen atom, halo-C₁₋₆ alkyl, N(R^{5G})₂, CN, NO₂, C(R^{6G})₃, CON(R^{5G})₂, OR^{4G}, SR^{4G} or S(O)_{nG}R^{7G},
R^{4G} is H, lower alkyl, lower alkenyl, lower alkynyl, CHF₂ or CF₃,
R^{5G} is R^{4G}, phenyl or benzyl, or two R^{5G} in combination with an at most six-membered ring containing carbon atoms and at most two hetero atom(s) selected from O, N and S,
R^{6G} is H, F, CF₃ or lower alkyl, or two R^{6G} may be taken together and represent an at most six-membered ring containing carbon atoms and 0 to 2 hetero atom(s) selected from O, N and S,
R^{7G} is lower alkyl, lower alkenyl, lower alkynyl, CHF₂, CF₃, N(R^{5G})₂, Ph(R^{8G})₂ or CH₂Ph(R^{8G})₂,
R^{8G} is R^{4G}, OR^{4G}SR^{4G} or a halogen atom,
W^{G} is a three- to six-membered connecting chain containing 0 to 2 hetero atom(s) selected from O, N and S and the connecting chain may contain CO, S(O)_{mG}, C═C or acetylene, and may be further substituted with R^{9G},
R^{9G} is R^{2G}, lower alkenyl, lower alkynyl, OR^{4G} or SR^{4G},
Ar^{2G} is aryl or heteroaryl which may be substituted with R^{3G},
R^{10G} is R^{4G}, a halogen atom, N(R^{5G})₂, CN, NO₂, C(R^{6G})₃, OR^{4G}, SR^{4G} or S(O)_{nG}R^{7G},
X^{G} is a connecting group which is substituted at the ortho position to Ar^{2G} based on W^{G} and it contains 0 to 4 carbon atom(s) and at most one hetero atom selected from O, N and S, may contain CO, S(O)_{nG}, C═C or acetylene, and may be further substituted with R^{11G}, R^{11G} has the same meaning as R^{9G},
Q^{G} is a group selected from COOH, tetrazole, SO₃H, hydroxamic acid, CONHSO₂R^{12G}, and SO₂NHCOR^{12G},
R^{12G} is a group selected from CF₃, lower alkyl, lower alkenyl, lower alkynyl and Z^{G}Ar^{4G},
Z^{G} is 0 to 4 connecting chain(s) which may be substituted with R^{13G},
R^{13G} has the same meaning as R^{9G},
Ar^{4G} is aryl or heteroaryl which may be substituted with R^{14G},
R^{14G} is R^{10G} or NHCOMe), or a non-toxic salt thereof.

### (8) A Compound of Formula (H) Mentioned in the Specification of WO 02/72564

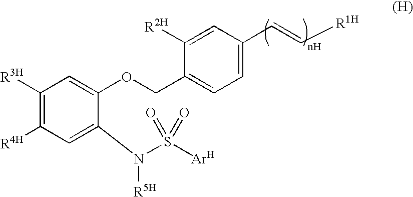

(wherein R^{1H} is COOH, 5-tetrazolyl, 5-oxo-1,2,4-oxadiazolyl, CH₂OH or 5-oxo-1,2,4-thiadiazolyl,
R^{2H} is hydrogen, methyl, methoxy or chloro,
R^{3H} and R^{4H} are the combination of (1) methyl and methyl, (2) methyl and chloro, (3) chloro and methyl, (4) trifluoromethyl and hydrogen, or
R^{3H} and R^{4H} are taken together to form cyclopentene, (6) cyclohexene or (7) benzene,
Ar^{H} is thiazolyl optionally substituted with methyl, pyridyl or 5-methyl-2-furyl,
nH is 0 or 1, but when R^{1H} is 5-tetrazolyl, 5-oxo-1,2,4-oxazolyl or 5-oxo-1,2,4-thiazolyl,
nH is 0), its alkyl ester or its non-toxic salt.
Besides, the followings are also preferably used as EP₁ receptor antagonists;
(9) the 2,3,6-substitute-4-pyrone compounds described in the specification of U.S. Pat. No. 4,132,847 as formula I,
(10) the N-alkenyl-3-hydroxybenzo[b]thiophene-2-carboxamide derivatives of formula I described in the specification of EP 160408
(11) the 8-chlorodibenz[b,f][1,4]oxazepine-10(11H)-carboxylic acid, 2-(sulfinyl- and sulfonyl-containing acyl)hydrazide compounds described as formula I in the specification of EP193822
(12) the 8-chlorodibenz[b,f][1,4]oxazepine-10 (11H)-carboxylic acid, 2-[(pheylthio-, pheylsulfinyl-, and phenylsulfonyl)alkanoyl]hydrazide compounds described as formula I in the specification of EP 218077,
(13) the cyclohept[b]indole alkanoic acid compounds described as formula I in the specification of U.S. Pat. No. 4,775,680,
(14) the tetrahydrocarbazole 1-alkanoic acid compound described in the specification of EP 300676 as formula I,
(15) the tricyclic heterocycle compounds of formula I described in the specification of EP 480641,
(16) the tricyclic heterocycle compounds of formula I described in the specification of EP 512399,
(17) the tricyclic heterocycle compounds of formula I described in the specification of EP 512400,
(18) the tricyclic heterocycle compounds of formula I described in the specification of EP 534667,
(19) the compound described in the specification of WO 93/071132,
(20) the substituted dibenzoxazepine compounds described as formula I in the specification of EP 539977,
(21) the substituted dibenzoxazepine or dibenzothiazepine compounds described as formula I in the specification of WO 93/13082,
(22) the 2-H and 3-alkoxy or hydroxy-8-substituted-dibenz[b,f]-[1,4]oxazepine-10(11)-carboxylic acid, substituted hydrazide compounds described as formula I in the specification of U.S. Pat. No. 5,281,590,
(23) the 1-, 2-, 3-, 4-, 5-, 6-, 7-, 8-, and/or 9-substituted dibenzoxazepine compounds described in the specification of U.S. Pat. No. 5,304,644,
(24) the 2-, 3-, 5-, 8-, 10-, and/or 11-substituted dibenzoxazepine compounds described as formula I in the specification of U.S. Pat. No. 5,324,722,
(25) the squaric acid derivatives of substituted dibenzoxazepine compounds described in the specification of U.S. Pat. No. 5,354,746 as formula I,
(26) the 2-, 3-, 4-, 5-, 6-, 7-, 8-, and/or 10-substituted dibenzoxazepine and dibenzthiazepine compounds described in the specification of U.S. Pat. No. 5,354,747 as formula I,
(27) the aryl substituted dibenzoxazepine compounds described in the specification of U.S. Pat. No. 5,420,270 as formula I,
(28) the substituted dibenzoxazepine and dibenzthiazepine carbamate compounds described in the specification of U.S. Pat. No. 5,441,950 as formula I,
(29) the substituted dibenzoxazepine compounds described in the specification of EP 694546 as formula I,
(30) the aromatic amino ether compounds described as formula I in the specification of WO 96/03380,
(31) the substituted dibenzoxazepine and dibenzthiazepine carbamate compounds described as formula I in the specification of U.S. Pat. No. 5,504,077,
(32) the compounds (D-X-A-Z-B—R′) described as formula I in the specification of WO 96/11902,
(33) the compound (D-O-A-C(R3)H—N(R2)-B—R1 described as formula I in the specification of EP 752421,
(34) the compound described in the specification of WO 97/00864,
(35) the compound described in the specification of WO 2003/43655,
(36) the benzenesulfonamide compounds described in the specification of EP 878465 as formula I,
(37) the N-phenylarylsulfonamide compounds described in the specification of WO 2002/72564 as formula I,
(38) the compounds described in the specification of WO 2002/72098 as formula I as and formula II and as formula III and as formula IV and as formula V,
(39) the compounds described in the specification of WO 2002/72145,
(40) the compounds described in the specification of WO 2000/69465
(41) the 2-((2-alkoxy)-phenyl)-cyclopent-1-enyl) aromatic carbo and heterocyclic acid and derivative compounds described as formula I in the specification of WO 2003/84917,
(42) the compounds described in the specification of WO 2003/101959 as formula I, and
(43) the compounds described in the specification of WO 2004/83185 as formula Ia or Ib,

Among the compounds mentioned in the above specifications, preferred ones are compounds which bind to an EP₁ receptor showing an antagonism and more preferably, the compounds which specifically bind to an EP₁ receptor showing an antagonism. Still more preferred ones are
[1] (5Z)-6-[(2R,3S)-3-({[(4-chloro-2-methylphenyl)sulfonyl]amino}methyl)bicycle [2.2.2]oct-2-yl]hex-5-enoic acid (mentioned in Example 2C of the specification of EP 878465),
[2] (2E)-3-(4-{[2-[(2-furylsulfonyl)(isobutyl)amino]-5-(trifluoromethyl)phenoxy]methyl}phenyl)acrylic acid,
[3] 4-{[2-{isopropyl[(5-methyl-2-furyl)sulfonyl]amino}-5-(trifluoromethyl)phenoxy]methyl}benzoic acid, and
[4] 4-{[(6-{isobutyl[(4-methyl-1,3-thiazol-2-yl)sulfonyl]amino}-2,3-dihydro-1H-inden-5-yl)oxy]methyl}benzoic acid.

The compounds shown in the above (1) to (43) can be manufactured by the method mentioned in each of the corresponding specifications of the International Publications, U.S. Patents or European Publications.

Preferably the EP₁ receptor antagonists will be more than 10 fold selective over the EP₂, EP₃, and EP₄ receptors respectively, more preferably more than 100 fold selective over the EP₂, EP₃, and EP₄ receptor respectively.

In addition, EP₁ receptor antagonists include, for example, SC-51322 (Hallinan et al (1994) Bioorg Med Chem Lett 4, 509-514), SC-19220 and analogoues thereof as discussed in Hallinan et al ((1993) J. Med. Chem. 36, 3293-3299), or SC-51089 and analogues thereof as discussed in Hallinan et al. ((1996) J. Med. Chem. 39, 609-613), ZD-4953 and analogues thereof (Ruel et al. Bioorg. Med. Chem Lett 9, 2699-2704) and others.

In the present specification, 4-({(1R,2R,3R)-3-hydroxy-2-[(1E,3S,5S)-3-hydroxy-5-methylnon-1-enyl]-5-oxocyclopentyl}acetyl)cyclohexanecarboxylic acid was used as EP₁ receptor agonist (compound A), and (2E)-3-(4-{[2-[(2-furylsulfonyl)(isobutyl)amino]-5-(trifluoromethyl)phenoxy]methyl}phenyl)acrylic acid was used as an EP₁ receptor antagonist (compound B).

EP₁ receptor antagonists have protective effect of cell-death induced by excitory amino acid-induced neuropathy, and therefore, it is useful for the prevention, treatment and/or inhibition of progress or protection of neurons in mammals (e.g., humans, non-human animals, e.g. monkeys, sheep, cattle, horses, dogs, cats, rabbits, rats, mice, etc.

Neurodegenerative diseases are not limited by the cause of the disease, but include all diseases accompanied by degeneration of neurons. Neurons include all ones in the live body, for example, central nervous system (e.g. brain, spinal cord, etc.), peripheral nervous system (e.g. neurons, spinal cord nerves, etc.). Neurodegenerative diseases are preferably, for example, diseases of central nervous system, for example, Parkinson's diseases, Parkinson syndrome, Alzheimer's diseases, dementia (e.g. vascular dementia, familial dementia, parieto-temporal dementia, senile dementia, AIDS dementia complex, Alzheimer dementia, etc.), Down's syndrome, amyotrophic lateral sclerosis, familial amyotrophic lateral sclerosis, progressive supranuclear palsy, Huntington's disease, spinocerebellar ataxia, dentatorubropallidoluysian atrophy, olivopontocerebellar atrophy, corticobasal degeneration, diffuse Lewy body disease, striatonigral degeneration, choreoathetosis, dystonia, Meige syndrome, late cerebellar cortical atrophy, familial spastic paraplegia, motor nerve disease, Machado-Joseph disease, Pick's disease, cerebral apoplexy (e.g. cerebral hemorrhage (e.g. hypertensive intracerebral hemorrhage etc.), cerebral infarction (e.g. cerebral thrombosis, cerebral embolism, etc.), temporary ischemic attack, subarachnoid hemorrhage, etc.), post-cerebrospinal traumatic neurological dysfunction, demyelinating disease (e.g. multiple sclerosis, Guillain-Barre syndrome, acute disseminated encephalomyelitis, acute parencephalitis, acute transverse myelitis, etc.), cerebral tumor (e.g. astrocytosis etc.), encephalomyelopathy accompanying infectious disease (e.g. cerebral meningitis).

Since an EP₁ receptor antagonist is useful for the prevention, treatment and/or inhibition of progress of neurodegenerative diseases, it is useful for treating and/or ameliorating tremor, rigidity, akinesia, bradykinesia, slow movement, postural reflex disorder, pulsion, gait disorder, depression, dysmnesia, amyotrophy, muscle weakness, dysfunctions of upper extremities, dysarthria, dysphagia, respiratory obstruction, palsy, paralysis, etc. which are symptoms of neurodegenerative diseases.

In the present invention, an EP₁ receptor antagonist may be administered together with dopamine receptor agonist (e.g. L-dopa, bromocriptine, pergolide, talipexole, pramipexol, cabergoline, amantadine, etc., monoamine oxidase inhibitor (e.g. safrazine, deprenyl, selegiline, remacemide, riluzole, etc., COMT inhibitor (e.g. Entacapone etc.), acetylcholine esterase inhibitor (e.g. donepezil hydrochloride, TAK-147, Rivastigmine, Galantamine, etc.), β-amyloid protein aggregation inhibitor (e.g. PTI-00703, ALZHEMED (NC-531), PPI-368, PPI-558, SKF-74652, etc.), β-secretase inhibitor, brain function activator, antioxidant, antithrombotic and astrocyte ameliorator (e.g. ONO-2506 (arundic acid) etc.), NMDA receptor antagonist (e.g., (+)-(1S,2S)-1-(4-hydroxy-phenyl)-2-(4-hydroxy-4-phenyl piperidino)-1-yl)-1-propanol, (1S,2S)-1-(4-hydroxy-3-methoxyphenyl)-2-(4-hydroxy-4-phenyl piperidino)-1-propanol, etc.), for the reinforcement of the efficacy of the compound.

### Isomers

In the present invention, all isomers are included unless specified. Isomers resulting from a double bond, a ring, a fused ring (E-, Z-, cis-, trans-isomer), isomers by the existence of asymmetric carbon atom etc. (R-, S-isomer, α-, β-position, enantiomer, diastereomer), optical isomers (D-, L-, d-, L-isomer), polar isomers by chromatographic separation (more polar, less polar isomer), equilibrium compound, rotatory isomer, a mixture thereof of arbitrary ratios are all included in the present invention.

In the present invention, as may be easily understood by those skilled in the art,
[symbol] indicates a single bond or a double bond, unless otherwise specified, the symbol:
[symbol] indicates that the substituent attached thereto is in front of the sheet (β-position),
[symbol] indicates that the substituent attached thereto is behind the sheet (α-position),
[symbol] and [symbol]
   indicates that the substituent attached thereto is in β-position or α-position or a mixture thereof.

### Salts and Solvates

The compounds used in the present invention may be converted into the corresponding salts by a conventional method. Non-toxic and water-soluble salts are preferable. Appropriate salts are described hereinafter; salts of alkali metals (e.g. potassium, sodium), salts of alkaline-earth metals (e.g. calcium, magnesium), ammonium salts, salts of pharmaceutically acceptable organic amines (tetramethyl ammonium, triethylamine, methylamine, dimethylamine, cyclopentylamine, benzylamine, phenethylamine, piperidine, monoethanolamine, diethanolamine, tris(hydroxymethyl)methylamine, lysine, arginine, N-methyl-D-glucamine etc.), acid-addition salts (inorganic salts (hydrochloride, hydrobromide, hydroiodide, sulfate, phosphate, nitrate, etc.), organic acid salts (acetate, trifluoroacetic acid, lactate, tartarate, oxalate, fumarate, malate, benzoate, citrate, methanesulfonate, ethanesulfonate, benzenesulfonate, toluenesulfonate, isethionate, glucronate, gluconate, etc.), etc.).

The compound of the present invention or a salt thereof may be converted into solvates by a conventional method. Solvates are preferably non-toxic, water-soluble. Solvates of the compound of the present invention include, solvates of water, alcoholic solvents (e.g. methanol, ethanol, etc.).

### Cyclodextrin Clathrates

The compound used in the present invention may be converted into cyclodextrin clathrates by the methods described in the specification of Japanese Kokoku No. 50-3362, Japanese Kokoku No. 52-31404 or Japanese Kokoku No. 61-52146. Conversion into their cyclodextrin clathrates serves to improve the stability, and solubility in water of the compounds, and therefore it is convenient in the use for pharmaceuticals.

### Prodrugs

The prodrug of the compound used in the present invention represents a compound which is converted to the active compound by enzymes or gastric acid in the live body. Prodrugs are an accepted means of delivering an active compound to a site in the body.

The following experiments, biological examples or appropriately improved methods thereof demonstrate efficacy. The compound of the present invention demonstrate excellent pharmacokinetics, e.g., the duration of serum half-life, gastrointestinal stability, oral absorbability, bioavailability, etc.

Although efficacy of the compounds having an EP₁ receptor antagonism to neurodegenerative diseases was proved by the following experiments, the present invention is not limited thereto. Compounds of the present invention are not limited to the substances having specific disclosed chemical structures but all compounds having an EP₁ receptor antagonism are included within the scope of the present invention.

The antagonists of the present invention can be provided in solid preparations for internal use, as well as in liquid preparations for internal use, for oral administration, as well as for injections. Preparations for external use include suppositories, and the like for parenteral administration.

Examples of the solid preparations for internal use include tablets, pills, capsules, dusts, granules, and the like. The capsules include hard capsules and soft capsules.

Such solid preparations for internal use are prepared by a formulation method commonly employed by using one or more active substances either as such or as a mixture with an excipient (lactose, mannitol, glucose, microcrystalline cellulose, starch, and the like), a binder (hydroxypropylcellulose, polyvinylpyrrolidone, magnesium metasilicate aluminate, and the like) a disintegrating agent (calcium cellulose glycolate, and the like), a lubricant (magnesium stearate, and the like), a stabilizer, and/or a dissolution aid (glutamic acid, aspartic acid, and the like). If necessary, it may be coated with a coating agent (sucrose, gelatin, hydroxypropylcellulose, hydroxypropylmethylcellulose phthalate, and the like). It may be coated with two or more layers. Moreover, capsules made of an absorbable material such as gelatin are within the scope of the invention.

The liquid preparations for internal use involve pharmaceutically acceptable solutions, suspensions, emulsions, syrups, elixirs and the like. Such a liquid preparation is prepared by dissolving, suspending, or emulsifying one or more active substances in a diluent commonly employed (purified water, ethanol, a mixture thereof, and the like). The liquid preparation may further contain any of a moistening agent, a suspending agent, an emulsifier, a sweetener, a flavor, a perfume, a preservative, a buffer and the like.

Injections for use in parenteral administration include sterile aqueous, suspension, emulsion and solid forms used by dissolving or suspending in solvent before use. For injections, one or more active substances is used by dissolving, suspending, or emulsifying in a solvent. Examples of solvents include distilled water for injection (pyrogen-free), physiological saline, vegetable oil, propylene glycol, polyethylene glycol, alcohols such as ethanol and combinations thereof. Furthermore, this injection may contain stabilizer, solubilizer (glutamic acid, aspartic acid, polysorbate 80 (registered trademark), and the like), suspending agent, emulsifying agent, analgesic agent, buffering agent, preservative agent, and the like. They may also be manufactured in the form of sterile solid forms, for example, freeze-dried products, which may be dissolved in sterile water or some other sterile diluent(s) for injection immediately before use.

The dosage forms of the parenteral administration preparations for external use involve ointments, gels, creams, fomentations, patches, liniments, atomized agents, inhalations, sprays, aerosols, nasal drops and the like. Such a preparation contains one or more active substances and is prepared by a publicly known method or in accordance with a formulation commonly employed.

Ointments are prepared in accordance with a publicly known formulation or a formulation commonly employed. For example, they are prepared by levigating or melting one or more active substances in a base. The ointment base is selected from among publicly known ones or those commonly employed. For example, use may be made of one base or a mixture of two or more thereof selected from higher fatty acids or higher fatty acid esters (adipic acid, myristic acid, palmitic acid, stearic acid, oleic acid, adipic acid esters, myristic acid esters, palmitic acid esters, stearic acid esters, oleic acid esters, and the like), waxes (beeswax, whale wax, ceresin, and the like), surfactants (polyoxyethylene alkyl ether phosphoric acid esters, and the like), higher alcohols (cetanol, stearyl alcohol, cetostaryl alcohol, and the like), silicone oils (dimethylpolysiloxane, and the like), hydrocarbons (hydrophilic vaseline, white vaseline, refined lanolin, liquid paraffin, and the like), glycols (ethylene glycol, diethylene glycol, propylene glycol, polyethylene glycol, macrogol, and the like), vegetable oils (castor oil, olive oil, sesame oil, turpentine oil, and the like), animal oils (mink oil, yolk oil, squalane, squalene, and the like), water, absorption promoters and skin irritation inhibitors. The ointments may further contain a humectant, a preservative, a stabilizer, an antioxidant, a flavor, and the like.

Gels are prepared in accordance with a publicly known formulation or a formulation commonly employed. For example, they are prepared by melting one or more active substances in a base. The gel base is selected from among publicly known ones or those commonly employed. For example, use may be made of one base or a mixture of two or more thereof selected from among lower alcohols (ethanol, isopropyl alcohol, and the like), gelling agents (carboxymethylcellulose, hydroxyethylcellulose, hydroxypropylcellulose, ethylcellulose, and the like), neutralizing agents (triethanolamine, diisopropanolamine, and the like), surfactants (polyethylene glycol monostearate, and the like), gums, water, absorption promoters and skin irritation inhibitors. The gels may further contain a preservative, an antioxidant, a flavor, and the like.

Creams are prepared in accordance with a publicly known formulation or a formulation commonly employed. For example, they are prepared by melting or emulsifying one or more active substances in a base. The cream base is selected from among publicly known ones or those commonly employed. For example, use may be made of one base or a mixture of two or more thereof selected from among higher fatty acid esters, lower alcohols, hydrocarbons, polyhydric alcohols (propylene glycol, 1,3-butylene glycol, and the like), higher alcohols (2-hexyldecanol, cetanol, and the like), emulsifiers (polyoxyethylene alkyl ethers, fatty acid esters, and the like), water, absorption promoters and skin irritation inhibitors. The creams may further contain a preservative, an antioxidant, a flavor, and the like.

Fomentations are prepared in accordance with a publicly known formulation or a formulation commonly employed. For example, they are prepared by melting one or more active substances in a base, kneading and then applying and spreading the kneaded matter on a substrate. The fomentation base is selected from among publicly known ones or those commonly employed. For example, use may be made of one base or a mixture of two or more thereof selected from among thickeners (polyacrylic acid, polyvinylpyrrolidone, acacia, starch, gelatin, methylcellulose, and the like), moistening agents (urea, glycerol, propylene glycol, and the like), fillers (kaolin, zinc oxide, talc, calcium, magnesium, and the like), water, dissolution aids, tackifiers and skin irritation inhibitors. The fomentations may further contain a preservative, an antioxidant, a flavor, and the like.

Patches are prepared in accordance with a publicly known formulation or a formulation commonly employed. For example, they are prepared by melting one or more active substances in a base and then applying and spreading on a substrate. The patch base is selected from among publicly known ones or those commonly employed. For example, use may be made of one base or a mixture of two or more thereof selected from among polymer bases, fats and oils, higher fatty acids, tackifiers and skin irritation inhibitors. The patches may further contain a preservative, an antioxidant, a flavor, and the like.

Liniments are prepared in accordance with a publicly known formulation or a formulation commonly employed. For example, they are prepared by dissolving, suspending or emulsifying one or more active substances in one or more media selected from water, alcohols (ethanol, polyethylene glycol, and the like), higher fatty acids, glycerol, soap, emulsifiers, suspending agents and the like. The liniments may further contain a preservative, an antioxidant, a flavor, and the like.

Atomized agents, inhalations, sprays and nasal drop may contain, in addition to a diluent commonly employed, a stabilizer such as sodium hydrogen sulfite, a buffer for imparting isotonicity, for example, an isotonic agent such as sodium chloride, sodium citrate or citric acid. Methods for producing sprays are described in detail in, for example, U.S. Pat. No. 2,868,691 and U.S. Pat. No. 3,095,355.

When nasal drops are administered, they are usually administered by spraying (atomizing) solution or powder containing drug into nasal cavity with a dedicated nasal drip apparatus or atomizer quantitatively.

Eye drops for parenteral administration may be in the form of liquid, suspension, emulsion, liquid dissolved in a solvent in use or ointment.

These eye drops are prepared by any known method. For example, one or more active substances are dissolved, suspended or emulsified in a solvent. As such a solvent for eye drops, there may be used sterilized purified water, physiological saline and other aqueous solvents or non-aqueous solvents for injection (vegetable oils, and the like), singly or in combination thereof. The eye drops may contain ones selected from an isotonic agent (sodium chloride, concentrated glycerin, and the like), a buffering agent (sodium phosphate, sodium acetate, and the like), a surfactant (Polysolvate 80 (trade name), Polyoxyl stearate 40, polyoxyethylene-hydrogenated castor oil, and the like), a stabilizer (sodium citrate, sodium edetate, and the like), a preservative (benzalconium chloride, paraben, and the like), and the like. The eye drops are sterilized at the final step or prepared by an aseptic process. Alternatively, an aseptic solid agent such as freeze-dried product which has previously been prepared may be rendered aseptic or dissolved in aseptic distilled water for injection or other solvents before use.

The dosages of inhalations for parenteral administration include aerosol, powders for inhalation or liquids for inhalation. The liquids for inhalation may be dissolved or suspended in water or the other appropriate solvent as needed.

Such inhalations are prepared in a known method. For example, liquids for inhalation are prepared by selecting proper additives from an antiseptic (benzalkonium chloride, p-aminobenzonic acid and the like), a coloring agent, a buffering agent (sodium phosphate, sodium acetate and the like), an isotonizing agent (sodium chloride, concentrated glycerin and the like), thickening agent (carboxyvinylpolymer and the like), or an accelerator of absorption, and the like, if necessary.

Powders for inhalation are prepared by selecting proper additives from a lubricant agent (such as stearin acid and the salt thereof), a binding agent) (starch, dextrin and the like), a diluting agent (lactose, cellulose and the like), a coloring agent, an antiseptic (benzalkonium chloride, p-aminobenzonic acid and the like), an accelerator of absorption, and the like, if necessary.

When liquids for inhalation are administered, sprays (atomizer, nebulizer, and the like) are usually used and when powders for inhalation are administered, inhalation administration apparatus for powder agents are usually used.

Other compositions for parenteral administration include suppositories for intrarectal administration and pessaries for vaginal administration which comprise one or more of the active substance(s) and may be prepared by methods known per se.

### Toxicity

The compounds used in the present invention have very low toxicity and so it was confirmed that they are safe for pharmaceutical use.

### BEST MODE FOR CARRYING OUT THE INVENTION

### Examples

The Examples below are carried out using standard techniques, which are well-known and routinely used by those skilled in the art; the examples illustrate but do not limit the invention.

### Example 1

### The Prostaglandin PGE₂ EP₁ Receptor Knockout Mice Attenuate Ischemia Reperfusion Injury

### Materials and Methods

This study was conducted in accordance with the National Institutes of Health guidelines for the use of experimental animals. Protocols were approved by the Institutional Animal Care and Use Committee at Johns Hopkins University. [C57BL/6] Male mice were divided into two groups: EP₁^{+/+} and EP₁^{-/-}, each having 12 animals. The animals of each group were sacrificed after 4 days of reperfusion and brains were dissected for TTC staining.

### Middle Cerebral Artery Occlusion and Reperfusion

Transient focal cerebral ischemia was induced by MCAO using an intraluminal filament technique. Under halothane anesthesia (2.0% for induction, 1.0% for maintenance), adult male mice (20-28 g at 8-10 weeks old) were ventilated with oxygen-enriched air via a nose cone. Body temperatures were maintained between 36.0-38.8° C. by a heating pad. Relative cerebral blood flow (CBF) was measured by laser-Doppler flowmetry (Moor instruments, Devon, England) with a flexible probe affixed to the skull over the parietal cortex supplied by the MCA (2 mm posterior and 6 mm lateral to bregma). Under aseptic conditions, the neck and carotid bifurcation were dissected. The common carotid artery was temporarily ligated and the external carotid artery was used as a stump. A small 7-0 Ethilon monofilament nylon (Ethicon, Inc., Somerville, N.J.) was covered with flexible silicone (Cutter Sil light universal hardener, Heraeus Kulzer GmbH, Hanau, Germany) and used as the filament to occlude the MCA. With an incision in the external carotid artery stump, the filament was advanced through the internal carotid artery to the origin of the MCA, documented by a decrease in laser-Doppler signal. The filament tip was left in position for 90 minutes of occlusion. After occlusion, the neck was closed with sutures, anesthesia was discontinued, and the animals were transferred to a controlled-temperature chamber to maintain body temperature at 37±5° C. At 90 minutes after occlusion, the mice were briefly anesthetized with halothane, and reperfusion was achieved by withdrawing the filament and reopening the middle cerebral artery, after which, the neck was sutured. The animals were returned to the controlled-temperature chamber for 6 hours and then returned to their cages and survived for 4 days.

### Measurement of Blood Gases and MABP

The femoral artery was cannulated for measurement of arterial blood gases and MABP at baseline, at ischemia, and then at 15-minute intervals for 90 minutes of ischemia and up to 60 minutes of reperfusion in separate cohorts of animals (n=5).

### Body Temperature

Body temperature was determined by using a rectal probe in separate cohorts of animals (n=5) at baseline, at ischemia, and at 15-minute intervals during 90 minutes of ischemia and MCAO and for up to 60 minutes of reperfusion.

### Quantification of Infarct Volume

Four days after reperfusion, mice were deeply anesthetized, and brains were harvested and sliced coronally into 2-mm thick sections and incubated with 1% 2,3,5-triphenyltetrazolium chloride (TTC) in saline for 30 min at 37° C. The area of infarcted brain, identified by the lack of TTC staining, was measured on the rostral and caudal surfaces of each slice and numerically integrated across the thickness of the slice to obtain an estimate of infarct volume (Sigma Scan Pro, Systat, Port Richmond, Calif.). Volumes from all five slices were summed to calculate total infarct volume over the entire hemisphere, expressed as a percentage of the volume of the contralateral hemisphere. Infarct volume was corrected for swelling by comparing the volumes in the ipsilateral and contralateral hemispheres. The corrected infarcted hemisphere is calculated as: volume of corrected infarcted hemisphere=volume of contralateral hemisphere−(volume of ipsilateral hemisphere−volume of infarcted hemisphere).

### Statistical Analysis

Data are expressed as means±SE. Multiple comparisons were analyzed by the ANOVA and Tukey's test, with significance at p value less than 0.05.

### Results

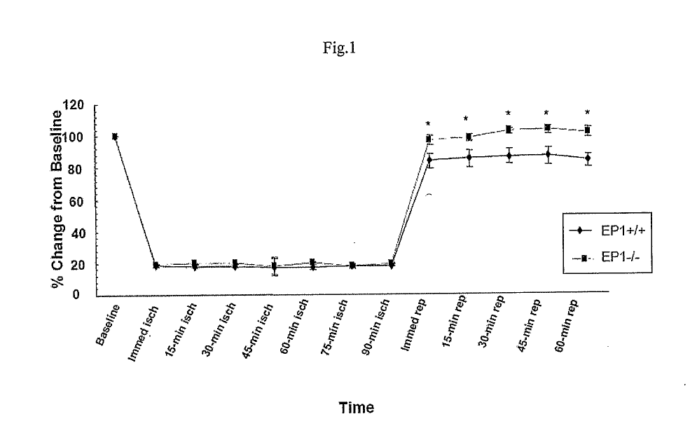

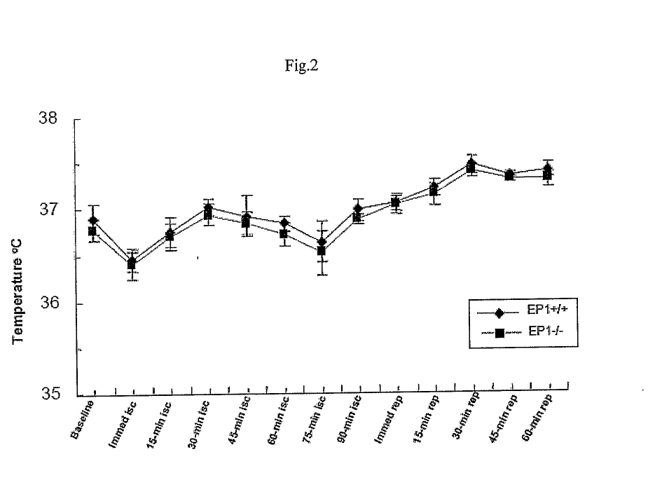

FIG. 1 shows the effect of MCAO on CBF as measured by laser-Doppler flowmetry (LDF). LDF drop was more than 80% in two different groups. No significant change in CBF was observed in the two groups during occlusion, but CBF increased significantly (P<0.05) in EP₁^{-/-} as compared to EP₁^{+/+} after reperfusion. FIG. 2 shows the effect of MCAO on body temperature, with no significant change in body temperature in either group.

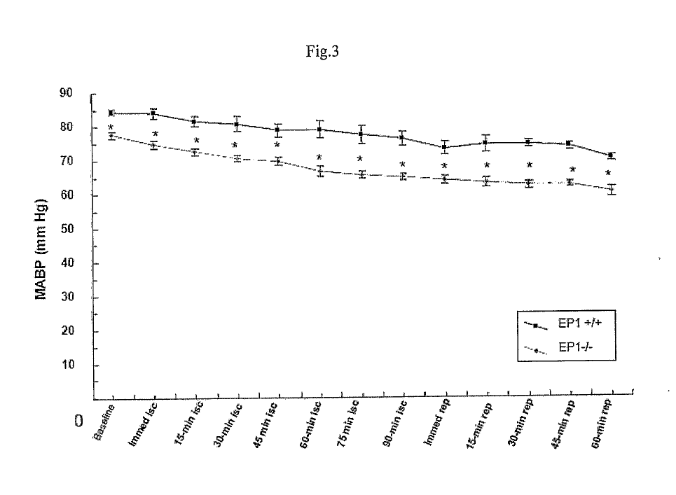

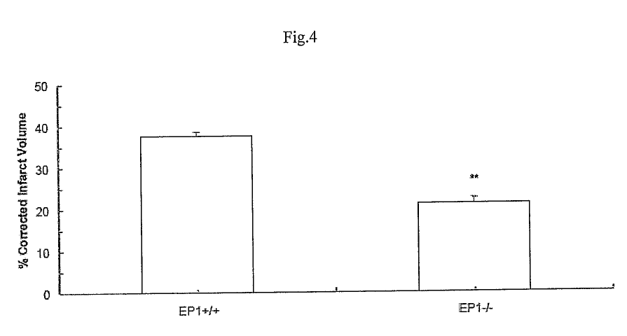

To assess the role of EP₁ in regulation of blood pressure, the functional consequences of EP₁ receptor inactivation were examined. MABP was measured using a femoral catheter and was lower in EP₁^{-/-} as compared to EP₁^{+/+} mice (P<0.05). We next analyzed blood pressure in separate groups of EP₁^{-/-} mice compared with male EP₁^{+/+} mice during MCAO and up to 1 hour of reperfusion. MABP in EP₁^{-/-} mice was significantly (P<0.05) lower than EP₁^{+/+} mice (FIG. 3). We hypothesized that genetic deletion of the EP₁ receptor in vivo would lead to decreased infarct volumes after stroke. A model of transient focal ischemia with MCAO followed by reperfusion was selected to test this hypothesis using male EP₁^{-/-} and EP₁^{+/+} mice (FIG. 4). Infarct volume decreased significantly (P<0.01) in infarct volume in male EP₁^{-/-} as compared to EP₁^{+/+}.

Table 1 shows physiologic measurements at baseline, during ischemia, and up to 1 hour after reperfusion (n=5 per genotype). Significant changes were observed in pO₂ level (P<0.05) between male EP₁^{-/-} and EP₁^{+/+} mice during MCAO, but significant difference was noted in pH, pCO₂ and Hb levels between the two groups.

TABLE 1
Effect of MCAO on Physiologic Parameters and Hemoglobin in
EP₁^{-/-} and EP₁^{+/+} Mice

| | | pH | pCO₂ | pO₂ | Hb |
|---|---|---|---|---|---|
| EP₁^{+/+} | Pre-ischemia | 7.36 ± 0.02 | 38.5 ± 1.08 | 105.1 ± 2.53 | |
| | 1-hr after | 7.33 ± 0.01 | 40.5 ± 1.12 | 130.6 ± 2.68 | |
| | ischemia | | | | |
| | Post-reperfusion | 7.34 ± 0.01 | 39.0 ± 1.12 | 106.7 ± 3.96 | 10.7 ± 0.25 |
| EP₁^{-/-} | Pre-ischemia | 7.373 ± 0.03 | 40.4 ± 1.0 | 104.2 ± 2.45 | |
| | 1-hr after | 7.331 ± 0.01 | 40.9 ± 1.16 | 113.8 ± 1.87* | |
| | ischemia | | | | |
| | Post-reperfusion | 7.347 ± 0.02 | 41.7 ± 1.17 | 108.8 ± 3.620 | 10.8 ± 0.18 |

Significance was determined by ANOVA followed by Tukey's test.
*P < 0.05 vs. EP₁^{+/+} mice.

### Example 2

### The Effect of an EP₁ Receptor Antagonist in the Treatment of Neurodegenerative Diseases, Using Wildtype and EP₁−/− Mice

### Materials and Methods

### Chemicals:

Unless stated otherwise, all chemicals were purchased from Sigma Co. (St. Louis, Mo.).

compound A (EP₁ receptor agonist):
(4-({(1R,2R,3R)-3-hydroxy-2-[(1E,3S,5S)-3-hydroxy-5-methylnon-1-enyl]-5-oxocyclopentyl}acetyl)cyclohexanecarboxylic acid) was prepared according to a method described in JP11-322709 and compound B (EP₁ receptor antagonist):
((2E)-3-(4-{[2-[(2-furylsulfonyl)(isobutyl)amino]-5-(trifluoromethyl)phenoxy]methyl}phenyl)acrylic acid) was prepared according to a method described in the specification of WO 02/72564.

### Mice:

Following protocols approved by the Institutional Animal Care and Use Committee of Johns Hopkins University, adult male C57BL/6 mice (Charles River, Wilmington, Mass.) and EP₁^{-/-}, weighing 20-25 g were used in this study.

### Treatment of Mice:

Weight and rectal temperature of each mouse was recorded before the surgical procedure. The mouse was subjected to intracerebroventricular (ICV) injection of vehicle or freshly prepared compound A or compound B followed by unilateral intrastriatal injection of NMDA, prepared in phosphate buffer saline (pH˜7.2) (J. Neurosci. 1997, 17, 6908-6917). In brief, each mouse was anesthetized, maintained at 1.0% halothane, and mounted on a stereotaxic frame (Stoelting Co., Wood Dale, Ill.). Varying body-weight (BW) doses of compound A (0.1 nmol/kg BW, n=8; 1 nmol/kg BW, n=12; 10 nmol/kg BW, n=8) or compound B (0.1 nmol/kg BW, n=8; 1 nmol/kg BW, n=12; 10 nmol/kg BW, n=9) were injected into the right lateral ventricle (stereotaxic coordinates PA-0.5 mm, lateral-1.0 mm from bregma, and ventral-2.5 mm relative to dura) over 30 sec, with the help of 1-μL Hamilton® syringe, and the needle was left in place for an additional 2 min. Twenty minutes later, 0.3 μl of 50 mM NMDA was injected into the right striatum over 2 min and the needle was left in place for additional 5 min. The NMDA-lesion control group (n=12) received 0.3 μL NMDA, and the vehicle-NMDA control group (n=11) received 0.2 μL vehicle ICV, followed by 0.3 μL NMDA in the striatum. Sham control and vehicle control groups were treated similarly with saline. After injections, mice were placed in a thermoregulated chamber maintained at 31° C. and returned to their cages after full recovery from anesthesia. Throughout the experimental procedure, rectal temperature of mice was monitored and maintained at 37.0±0.5° C.

### Assessment of Lesion Volume:

At 48 h after perfusion and fixation, weight and rectal temperature were recorded and brains were harvested and immediately frozen in 2-methyl butane (pre-cooled over dry ice). Brain sections were cut on a cryostat and stained with cresyl violet to estimate lesion volume. Images of the brain sections were taken and analyzed with the help of SigmaScan Pro 5.0 (Systat, Inc., Point Richmond, Calif.).

### Statistical Analysis:

Data were analyzed using SigmaStat 2.0 (Systat), and significance level was set at p<0.05. Statistical analysis was performed by Student's t-test. All data are reported as means±s.d.

### Results

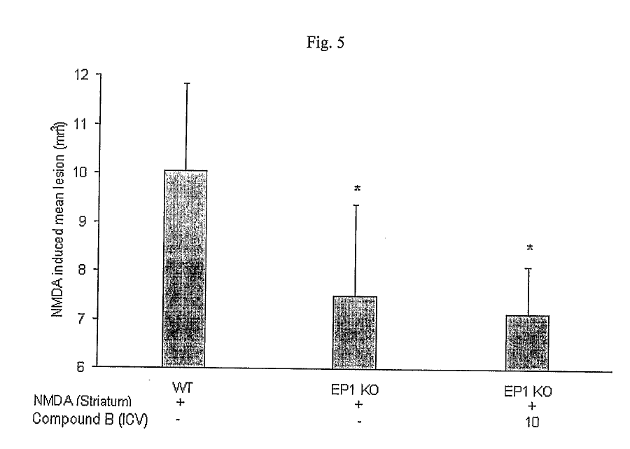

Genetic deletion of EP₁ receptor is beneficial in protecting the brain from excitotoxicity. EP₁^{-/-} mice were found to be less vulnerable to NMDA-induced toxicity as compared to C57BL/6 wildtype mice (FIG. 5). The ICV treatment of EP₁^{-/-} mice with compound B (EP₁ receptor antagonist) produced no significant difference as compared to the EP₁^{-/-} group (FIG. 5), demonstrating the selectivity of the drug toward EP₁ receptor and confirming the genetic deletion of EP₁ receptor in these mice.

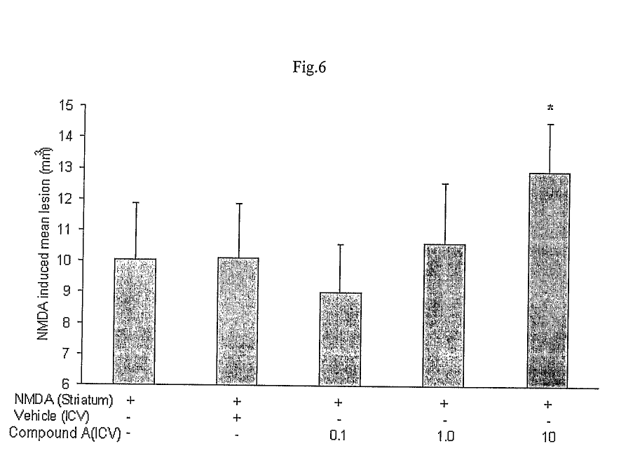

NMDA-mediated brain lesion is aggravated by compound A. ICV pretreatment of mice with compound A aggravated the brain injury caused by NMDA injection. Body-weight doses of 0.1 and 1 nmol/kg caused non-significant increases in brain lesion volume, whereas 10 nmol/kg BW caused a significant increase in brain lesion volume (FIG. 6).

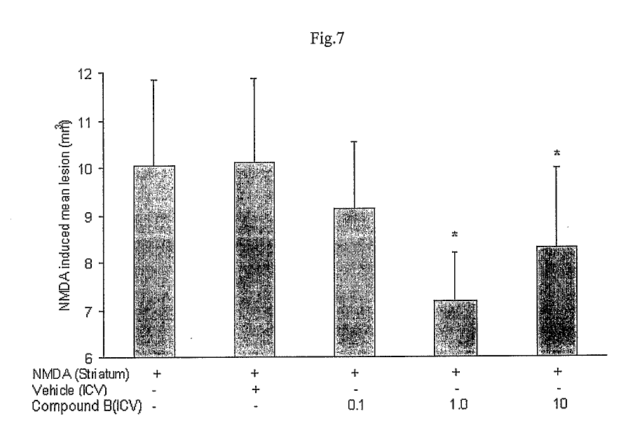

NMDA-mediated brain lesion is attenuated by compound B. Injection of 50 mM NMDA produced an apparent lesion in the ipsilateral striatum, and ICV pretreatment with compound B (1 nmol/kg BW) reduced the lesion volume. Lesion volumes in compound B-pretreated groups were compared with the NMDA-lesion group and vehicle-NMDA group. Pretreatment with compound B (1 and 10 nmol/kg BW) significantly decreased the NMDA-induced lesion volume (FIG. 7). No significant protection was observed with a dose of 0.1 nmol/kg BW, while a 10-nmol/kg BW dose showed slight, but not significant, toxicity as compared to 1 mmol/kg BW group. No lesion was observed in the sham-control or vehicle-control groups.

### Formulation Example 1

The following components (1) to (4) were admixed by a conventional method, punched out by a conventional method to give 100 tablets each containing 5 mg of active ingredient.

(1) 3-methyl-4-[2-[N-isobutyl-N-(5-methyl-2-furylsulfonyl)amino]-4,5-dimethyl phenoxymethyl]benzoic acid 500 mg
(2) calcium carboxymethylcellulose (disintegrating agent) 200 mg
(3) magnesium stearate (lubricant) 100 mg
(4) microcrystalline cellulose 9.2 g

### Formulation example 2

The following components (1) to (3) were admized and the resulting solution was sterilized in a conventional method, placed 3 ml portions into 5 ml ampoules to obtain 100 ampoules each containing 5 μg of the active ingredient.

(1) 3-methyl-4-[2-[N-isobutyl-N-(5-methyl-2-furylsulfonyl)amino]-4,5-dimethylphenoxymethyl]benzoic acid 500 mg
(2) mannitol 50 g
(3) distilled water 100 ml

What is claimed is:

1. A method for the prevention and/or treatment of neurodegenerative diseases in a patient comprising:
administering to the patient a therapeutically effective amount of a compound which antagonizes an EP₁ receptor.

2. The method according to claim 1, wherein the compound is selected from the group consisting of
(A) a compound of formula (A) 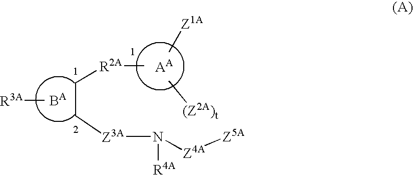
(wherein 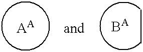
each independently is a C₅₋₁₅ carbon ring or a five- to seven-membered hereto ring having 1 or 2 oxygen, sulfur or nitrogen atom(s),
Z^{1A} is —COR^{1A}, —C₁₋₄ alkylene-COR^{1A}, —CH═CH—COR^{1A}, —C≡C—COR^{1A}, —O—C₁₋₃ alkylene-COR^{1A} (in each formula R^{1A} is hydroxyl group, C₁₋₄ alkoxy or a group represented by a formula NR⁶R^{7A} (in the formula R^{6A} and R^{7A} each independently is a hydrogen atom or C₁₋₄ alkyl) or —C₁₋₅ alkylene-OH,
Z^{2A} is a hydrogen, C₁₋₄ alkyl, C₁₋₄ alkoxy, nitro, halogen, trifluoromethyl, trifluoromethoxy, a hydroxyl group or a group represented by the formula COR^{1A} (in the formula R^{1A} has the same meaning as hereinbefore defined),
Z^{3A} is a single bond or C₁₋₄ alkylene,
Z^{4A} is SO₂ or CO,
Z^{5A} is
(1) C₁₋₈ alkyl, C₂₋₈ alkenyl, C₂₋₈ alkynyl,
(2) phenyl, C₃₋₇ cycloalkyl, a five- to seven-membered hetero ring having one or two oxygen, sulfur or nitrogen atom(s),
(3) phenyl or C₃₋₇ cycloalkyl-substituted C₁₋₄ alkyl, C₂₋₄ alkenyl or C₂₋₄ alkynyl,
(in the above (2) and (3), phenyl, C₃₋₇ cycloalkyl and a five- to seven-membered hetero ring having one or two oxygen, sulfur or nitrogen atom(s) may be substituted with one to five R^{5A} group(s) (each of plural R^{5A} independently is a hydrogen atom, C₁₋₆ alkyl, C₁₋₆ alkoxy, C₁₋₆ alkylthio, nitro, halogen, trifluoromethyl, trifluoromethoxy or a hydroxyl group)),
R^{2A} is CONR^{8A}, NR^{8A}CO, CONR^{8A}—C₁₋₄ alkylene, C₁₋₄ alkylene-CONR^{8A}, NR^{8A}CO—C₁₋₄ alkylene, C₁₋₄ alkylene-NR^{8A}CO, C₁₋₃ alkylene-CONR^{8A}—C₁₋₃ alkylene,
C₁₋₃ alkylene-NR^{8A}CO—C₁₋₃ alkylene (in each formula, R^{8A} is a hydrogen atom or C₁₋₄ alkyl), O, S, NZ^{6A} (in each formula, Z^{6A} is a hydrogen atom or C₁₋₄ alkyl), Z^{7A}-C₁₋₄ alkylene, C₁₋₄ alkylene-Z^{7A}, C₁₋₃ alkylene-Z^{7A}-C₁₋₃ alkylene (in the formulae, Z^{7A} is O, S or a group represented by the formula NZ^{6A} (in the formula, Z^{6A} has the same meaning as defined above)), CO, CO—C₁₋₄ alkylene, C₁₋₄ alkylene-CO, C₁₋₃ alkylene-CO—C₁₋₃ alkylene, C₂₋₄ alkylene, C₂₋₄ alkenylene or C₂₋₄ alkynylene,
R^{3A} is a hydrogen atom, C₁₋₆ alkyl, C₁₋₆ alkoxy, C₁₋₆ alkylthio, nitro, halogen, trifluoromethyl, trifluoromethoxy, hydroxy or hydroxymethyl,
R^{4A} is
(1) a hydrogen atom,
(2) C₁₋₈ alkyl, C₂₋₈ alkenyl, C₂₋₈ alkynyl,
(3) C₁₋₆ alkyl substituted with one or two group(s) selected from the group consisting of COOZ^{8A}, CONZ^{9A}Z^{10A}, OZ^{8A} (in each group, Z^{8A}, Z^{9A} and Z^{10A} each independently is hydrogen atom or C₁₋₄ alkyl), C₁₋₄ alkoxy-C₁₋₄ alkoxy,
(4) C₃₋₇ cycloalkyl,
(5) phenyl or C₃₋₇ cycloalkyl-substituted C₁₋₄ alkyl, C₂₋₄ alkenyl or C₂₋₄ alkynyl, (phenyl and C₃₋₇ cycloalkyl in the above (4) and (5) may be substituted with one to five R^{5A} group(s) (R^{5A} has the same meaning as defined above))
and n^{A} and t^{A} each independently is an integer of 1 to 4,
wherein
(1) R^{2A} and Z^{3A} each binds to only 1- and 2-position of 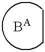
and
(2) when 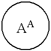
is a benzene ring and (Z^{2A})ₜ^{A} is not COR^{1A}, then Z^{1A} binds to only 3- or 4-position of the benzene ring) or a non-toxic salt thereof;
(B) a compound of formula (B) 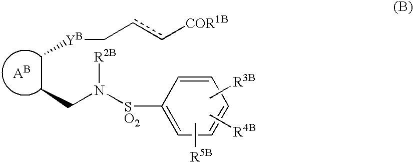
(in the formula, 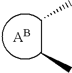
is a group represented by 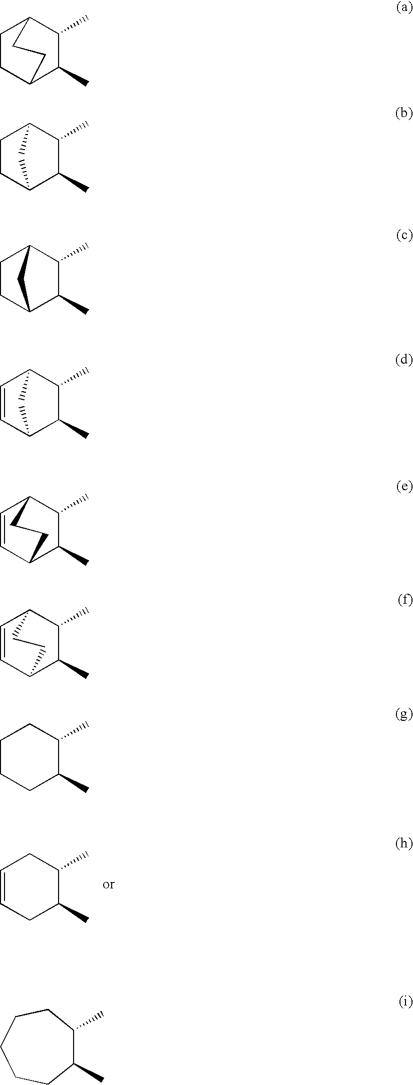
R^{1B} is hydroxy, C₁₋₄ alkoxy or alkoxy or a group represented by formula NR^{6B}R^{7B} (in the formula, R^{6B} and R^{7B} each independently is a hydrogen atom or C₁₋₄ alkyl group),
R^{2B} is a hydrogen atom or C₁₋₄ alkyl group,
R^{3B} and R^{4B} are a C₁₋₄ alkyl group, a halogen atom or trifluoromethyl group,
R^{5B} is a hydrogen atom, a C1-4 alkyl group, a halogen atom or trifluoromethyl group,
Y^{B} is cis-vinylene or trans-vinylene and
a symbol [symbol] is a single bond or a double bond,
wherein when 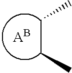
is a formula 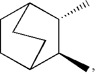
R^{1B} is hydroxy or C₁₋₄ alkoxy group, R^{2B} is a hydrogen atom, Y^{B} is cis-vinylene and the symbol [symbol] is a single bond, then 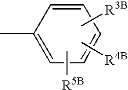
is not 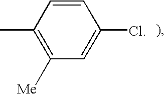
or a non-toxic salt thereof or a cyclodextrin clathrate thereof; and
(C) a compound of formula (H) 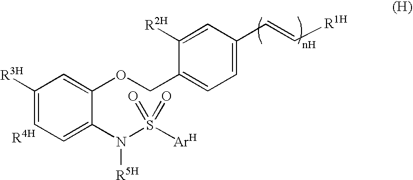
(wherein R^{1H} is COOH, 5-tetrazolyl, 5-oxo-1,2,4-oxadiazolyl, CH₂OH or 5-oxo-1,2,4-thiadiazolyl,
R^{2H} is hydrogen, methyl, methoxy or chloro,
R^{3H} and R^{4H} are the combination of (1) methyl and methyl, (2) methyl and chloro, (3) chloro and methyl, (4) trifluoromethyl and hydrogen, or
R^{3H} and R^{4H} are taken together to form cyclopentene, (6) cyclohexene or (7) benzene,
Ar^{H} is thiazolyl optionally substituted with methyl, pyridyl or 5-methyl-2-furyl,
nH is 0 or 1, but when R^{1H} is 5-tetrazolyl, 5-oxo-1,2,4-oxazolyl or 5-oxo-1,2,4-thiazolyl,
nH is 0); its alkyl ester; or its non-toxic salt.

3. The method of claim 1 wherein the EP₁ receptor antagonist is selected from the group consisting of:
(1) (5Z)-6-[(2R,3S)-3-({[(4-chloro-2-methylphenyl)sulfonyl]amino}methyl)bicyclo[2.2.2]oct-2-yl]hex-5-enoic acid,
(2) (2E)-3-(4-{[2-[(2-furylsulfonyl)(isobutyl)amino]-5-(trifluoromethyl)phenoxy]methyl}phenyl)acrylic acid,
(3) 4-{[2-{isopropyl[(5-methyl-2-furyl)sulfonyl]amino}-5-(trifluoromethyl)phenoxy]methyl}benzoic acid
(4) 4-{[(6-{isobutyl[(4-methyl-1,3-thiazol-2-yl)sulfonyl]amino}-2,3-dihydro-1H-inden-5-yl)oxy]methyl}benzoic acid,
a salt thereof, an N-oxide or solvate thereof, a prodrug thereof, and a cyclodextrin clathrate thereof.

4. The method of claim 1, wherein the neurodegenerative disease is selected from the group consisting of Parkinson's disease, Parkinson syndrome, Alzheimer's disease, dementia, amyotrophic lateral sclerosis, and cerebral stroke.

5. The method of claim 1 wherein an effective dose is administered for ameliorating a symptom selected from the group consisting of tremor, rigidity, akinesia, bradykinesia, slow movement, postural reflex disorder, pulsion, gait disorder, depression, dysmnesia, amyotrophy, muscle weakness, dysfunctions of upper extremities, dysarthria, dysphagia, respiratory obstruction, palsy, and paralysis.

6. A pharmaceutical composition for the treatment and/or prevention of neurodegenerative diseases comprising: a compound which antagonizes an EP₁ receptor.

7. The pharmaceutical composition according to claim 6, wherein the neurodegenerative disease is selected from the group consisting of Parkinson's disease, Parkinson syndrome, Alzheimer's disease, dementia, amyotrophic lateral sclerosis, and cerebral stroke.

8. The pharmaceutical composition according to claim 6 in an effective concentration for ameliorating a symptom selected from the group consisting of tremor, rigidity, akinesia, bradykinesia, slow movement, postural reflex disorder, pulsion, gait disorder, depression, dysmnesia, amyotrophy, muscle weakness, dysfunctions of upper extremities, dysarthria, dysphagia, respiratory obstruction, palsy, and paralysis.

9. The pharmaceutical composition according to claim 6 wherein the EP₁ receptor antagonist is admixed with a substance selected from the group consisting of: dopamine receptor agonist, a monoamine oxidase inhibitor, a COMT inhibitor, an acetylcholine esterase inhibitor, a β-amyloid protein aggregation inhibitor, a β-secretase inhibitor, a brain function activator, an antioxidant, an antithrombotic, an astrocyte ameliorator, and an NMDA receptor antagonist.

10. The pharmaceutical composition of claim 6 wherein the EP₁ receptor antagonist is selected from the group consisting of:
(1) (5Z)-6-[(2R,3S)-3-({[(4-chloro-2-methylphenyl)sulfonyl]amino}methyl)bicyclo[2.2.2]oct-2-yl]hex-5-enoic acid,
(2) (2E)-3-(4-{[2-[(2-furylsulfonyl)(isobutyl)amino]-5-(trifluoromethyl)phenoxy]methyl}phenyl)acrylic acid,
(3) 4-{[2-{isopropyl[(5-methyl-2-furyl)sulfonyl]amino}-5-(trifluoromethyl)phenoxy]methyl}benzoic acid
(4) 4-{[(6-{isobutyl[(4-methyl-1,3-thiazol-2-yl)sulfonyl]amino}-2,3-dihydro-1H-inden-5-yl)oxy]methyl}benzoic acid,
a salt thereof, an N-oxide or solvate thereof, a prodrug thereof, and a cyclodextrin clathrate thereof.

11. Use of an EP₁ receptor antagonist for the manufacture of a medicament for the prevention, treatment and/or inhibition of progress of neurodegenerative disease.

12. A method for the inhibition of progress of a neurodegenerative diseases in a patient comprising administering a therapeutically effective amount of a compound having an antagonism to an EP₁ receptor.

13. The pharmaceutical composition according to claim 6, wherein the compound is selected from the group consisting of
(A) a compound of formula (A) 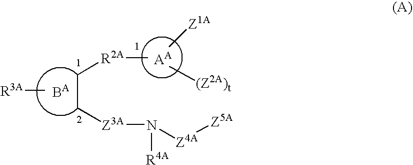
(wherein 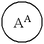
and 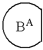
each independently is a C₅₋₁₅ carbon ring or a five- to seven-membered hereto ring having 1 or 2 oxygen, sulfur or nitrogen atom(s),
Z^{1A} is —COR^{1A}, —C₁₋₄ alkylene-COR^{1A}, —CH═CH—COR^{1A}, —C≡C—COR^{1A}, —O—C₁₋₃ alkylene-COR^{1A} (in each formula R^{1A} is hydroxyl group, C₁₋₄ alkoxy or a group represented by a formula NR^{6A}R^{7A} (in the formula R^{6A} and R^{7A} each independently is a hydrogen atom or C₁₋₄ alkyl) or —C₁₋₅ alkylene-OH,
Z^{2A} is a hydrogen, C₁₋₄ alkyl, C₁₋₄ alkoxy, nitro, halogen, trifluoromethyl, trifluoromethoxy, a hydroxyl group or a group represented by the formula COR^{1A} (in the formula R^{1A} has the same meaning as hereinbefore defined),
Z^{3A} is a single bond or C₁₋₄ alkylene,
Z^{4A} is SO₂ or CO,
Z^{5A} is
(1) C₁₋₈ alkyl, C₂₋₈ alkenyl, C₂₋₈ alkynyl,
(2) phenyl, C₃₋₇ cycloalkyl, a five- to seven-membered hetero ring having one or two oxygen, sulfur or nitrogen atom(s),
(3) phenyl or C₃₋₇ cycloalkyl-substituted C₁₋₄ alkyl, C₂₋₄ alkenyl or C₂₋₄ alkynyl,
(in the above (2) and (3), phenyl, C₃₋₇ cycloalkyl and a five- to seven-membered hetero ring having one or two oxygen, sulfur or nitrogen atom(s) may be substituted with one to five R^{5A} group(s) (each of plural R^{5A} independently is a hydrogen atom, C₁₋₆ alkyl, C₁₋₆ alkoxy, C₁₋₆ alkylthio, nitro, halogen, trifluoromethyl, trifluoromethoxy or a hydroxyl group)),
R^{2A} is CONR^{8A}, NR^{8A}CO, CONR^{8A}—C₁₋₄ alkylene, C₁₋₄ alkylene-CONR^{8A}, NR^{8A}CO—C₁₋₄ alkylene, C₁₋₄ alkylene-NR^{8A}CO, C₁₋₃ alkylene-CONR^{8A}—C₁₋₃ alkylene, C₁₋₃ alkylene-NR^{8A}CO—C₁₋₃ alkylene (in each formula, R^{8A} is a hydrogen atom or C₁₋₄ alkyl), O, S, NZ^{6A} (in each formula, Z^{6A} is a hydrogen atom or C₁₋₄ alkyl), Z^{7A}-C₁₋₄ alkylene, C₁₋₄ alkylene-Z^{7A}, C₁₋₃ alkylene-Z^{7A}-C₁₋₃ alkylene (in the formulae, Z^{7A} is O, S or a group represented by the formula NZ^{6A} (in the formula, Z^{6A} has the same meaning as defined above)), CO, CO—C₁₋₄ alkylene, C₁₋₄ alkylene-CO, C₁₋₃ alkylene-CO—C₁₋₃ alkylene, C₂₋₄ alkylene, C₂₋₄ alkenylene or C₂₋₄ alkynylene,
R^{3A} is a hydrogen atom, C₁₋₆ alkyl, C₁₋₆ alkoxy, C₁₋₆ alkylthio, nitro, halogen, trifluoromethyl, trifluoromethoxy, hydroxy or hydroxymethyl,
R^{4A} is
(1) a hydrogen atom,
(2) C₁₋₈ alkyl, C₂₋₈ alkenyl, C₂₋₈ alkynyl,
(3) C₁₋₆ alkyl substituted with one or two group(s) selected from the group consisting of COOZ^{8A}, CONZ^{9A}Z^{10A}, OZ^{8A} (in each group, Z^{8A}, Z^{9A} and Z^{10A} each independently is a hydrogen atom or C₁₋₄ alkyl), C₁₋₄ alkoxy-C₁₋₄ alkoxy,
(4) C₃₋₇ cycloalkyl,
(5) phenyl or C₃₋₇ cycloalkyl-substituted C₁₋₄ alkyl, C₂₋₄ alkenyl or C₂₋₄ alkynyl, (phenyl and C₃₋₇ cycloalkyl in the above (4) and (5) may be substituted with one to five R^{5A} group(s) (R^{5A} has the same meaning as defined above))
and n^{A} and t^{A} each independently is an integer of 1 to 4,
wherein
(1) R^{2A} and Z^{3A} each binds to only 1- and 2-position of 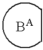
and
(2) when 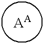
is a benzene ring and (Z^{2A})ₜ^{A} is not COR^{1A}, then Z^{1A} binds to only 3- or 4-position of the benzene ring) or a non-toxic salt thereof;
(B) a compound of formula (B) 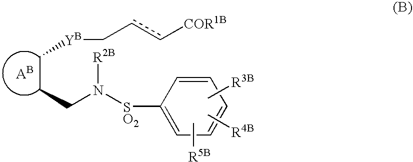
(in the formula, 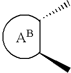
is a group represented by 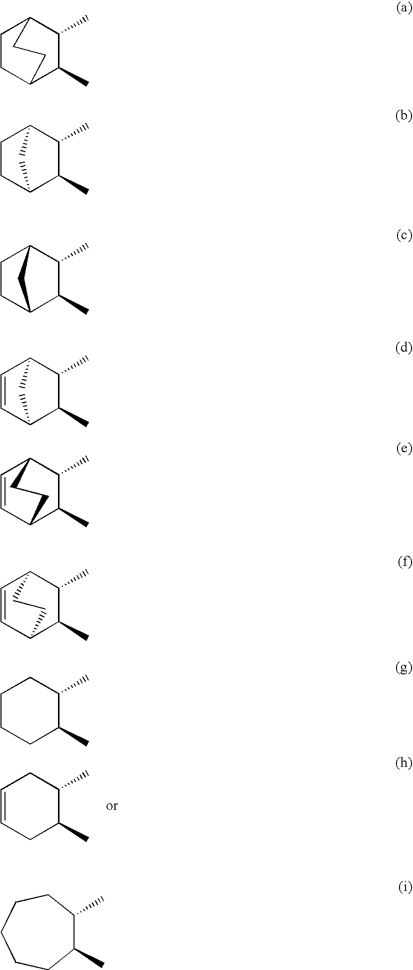
R^{1B} is hydroxy, C₁₋₄ alkoxy or alkoxy or a group represented by formula NR^{6B}R^{7B} (in the formula, R^{6B} and R^{7B} each independently is a hydrogen atom or C₁₋₄ alkyl group),
R^{2B} is a hydrogen atom or C₁₋₄ alkyl group,
R^{3B} and R^{4B} are a C₁₋₄ alkyl group, a halogen atom or trifluoromethyl group,
R^{5B} is a hydrogen atom, a C1-4 alkyl group, a halogen atom or trifluoromethyl group,
Y^{B} is cis-vinylene or trans-vinylene and
a symbol [symbol] is a single bond or a double bond,
wherein when 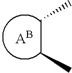
is a formula 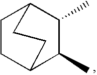
R^{1B} is hydroxy or C₁₋₄ alkoxy group, R^{2B} is a hydrogen atom, Y^{B} is cis-vinylene and the symbol [symbol] is a single bond, then 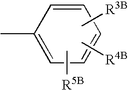
is not 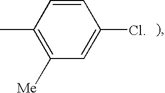
or a non-toxic salt thereof or a cyclodextrin clathrate thereof; and
(C) a compound of formula (H) 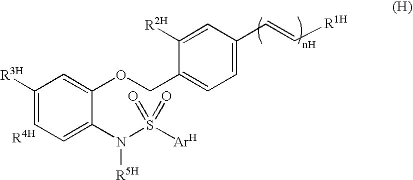
(wherein R^{1H} is COOH, 5-tetrazolyl, 5-oxo-1,2,4-oxadiazolyl, CH₂OH or 5-oxo-1,2,4-thiadiazolyl,
R^{2H} is hydrogen, methyl, methoxy or chloro,
R^{3H} and R^{4H} are the combination of (1) methyl and methyl, (2) methyl and chloro, (3) chloro and methyl, (4) trifluoromethyl and hydrogen, or
R^{3H} and R^{4H} are taken together to form cyclopentene, (6) cyclohexene or (7) benzene,
Ar^{H} is thiazolyl optionally substituted with methyl, pyridyl or 5-methyl-2-furyl,
nH is 0 or 1, but when R^{1H} is 5-tetrazolyl, 5-oxo-1,2,4-oxazolyl or 5-oxo-1,2,4-thiazolyl, nH is 0); its alkyl ester; and its non-toxic salt.

14. A method for protecting neurons induced by an excitory amino acid comprising contacting said neurons with an EP₁ receptor antagonist.
